SEQUENCE LISTING (141 sequences)

SEQ ID NO: 1 - protein, 101 aa, Human immunodeficiency virus type 1
MEPVDPKLEPWKHPGSQPKTACNNCYCKKCCFHCQVCFTKKGLGISYGRKKRRQRRRSPQDSETHQVSLSKQPASQPRGDPTGPKESKKKVERETETDPVD

SEQ ID NO: 2 - protein, 101 aa, Artificial Sequence
  Description of Artificial Sequence K51T-R52L mutant of Tat protein
MEPVDPKLEPWKHPGSQPKTACNNCYCKKCCFHCQVCFTKKGLGISYGRKTLRQRRRSPQDSETHQVSLSKQPASQPRGDPTGPKESKKKVERETETDPVD

SEQ ID NO: 3 - protein, 101 aa, Artificial Sequence
  Description of Artificial Sequence K51T-R55L mutant of Tat protein
MEPVDPKLEPWKHPGSQPKTACNNCYCKKCCFHCQVCFTKKGLGISYGRKTRRQLRRSPQDSETHQVSLSKQPASQPRGDPTGPKESKKKVERETETDPVD

SEQ ID NO: 4 - protein, 101 aa, Artificial Sequence
  Description of Artificial Sequence K51T-R57L mutant of Tat protein
MEPVDPKLEPWKHPGSQPKTACNNCYCKKCCFHCQVCFTKKGLGISYGRKTRRQRRLSPQDSETHQVSLSKQPASQPRGDPTGPKESKKKVERETETDPVD

SEQ ID NO: 5 - protein, 101 aa, Artificial Sequence
  Description of Artificial Sequence K51T-G79A mutant of Tat protein
MEPVDPKLEPWKHPGSQPKTACNNCYCKKCCFHCQVCFTKKGLGISYGRKTRRQRRRSPQDSETHQVSLSKQPASQPRADPTGPKESKKKVERETETDPVD

SEQ ID NO: 6 - protein, 101 aa, Artificial Sequence
  Description of Artificial Sequence K51T-K89L mutant of Tat protein
MEPVDPKLEPWKHPGSQPKTACNNCYCKKCCFHCQVCFTKKGLGISYGRKTRRQRRRSPQDSETHQVSLSKQPASQPRGDPTGPKESKLKVERETETDPVD

SEQ ID NO: 7 - protein, 101 aa, Artificial Sequence
  Description of Artificial Sequence K51T-E92Q mutant of Tat protein
MEPVDPKLEPWKHPGSQPKTACNNCYCKKCCFHCQVCFTKKGLGISYGRKTRRQRRRSPQDSETHQVSLSKQPASQPRGDPTGPKESKKKVQRETETDPVD

SEQ ID NO: 8 - protein, 101 aa, Artificial Sequence
  Description of Artificial Sequence R52L-R55L mutant of Tat protein
MEPVDPKLEPWKHPGSQPKTACNNCYCKKCCFHCQVCFTKKGLGISYGRKKLRQLRRSPQDSETHQVSLSKQPASQPRGDPTGPKESKKKVERETETDPVD

SEQ ID NO: 9 - protein, 101 aa, Artificial Sequence
  Description of Artificial Sequence R52L-R57L mutant of Tat protein
MEPVDPKLEPWKHPGSQPKTACNNCYCKKCCFHCQVCFTKKGLGISYGRKKLRQRRLSPQDSETHQVSLSKQPASQPRGDPTGPKESKKKVERETETDPVD

SEQ ID NO: 10 - protein, 101 aa, Artificial Sequence
  Description of Artificial Sequence R52L-G79A mutant of Tat protein
MEPVDPKLEPWKHPGSQPKTACNNCYCKKCCFHCQVCFTKKGLGISYGRKKLRQRRRSPQDSETHQVSLSKQPASQPRADPTGPKESKKKVERETETDPVD

SEQ ID NO: 11 - protein, 101 aa, Artificial Sequence
  Description of Artificial Sequence R52L-K89L mutant of Tat protein
MEPVDPKLEPWKHPGSQPKTACNNCYCKKCCFHCQVCFTKKGLGISYGRKKLRQRRRSPQDSETHQVSLSKQPASQPRGDPTGPKESKLKVERETETDPVD

SEQ ID NO: 12 - protein, 101 aa, Artificial Sequence
  Description of Artificial Sequence R52L-E92Q mutant of Tat protein
MEPVDPKLEPWKHPGSQPKTACNNCYCKKCCFHCQVCFTKKGLGISYGRKKLRQRRRSPQDSETHQVSLSKQPASQPRGDPTGPKESKKKVQRETETDPVD

SEQ ID NO: 13 - protein, 101 aa, Artificial Sequence
  Description of Artificial Sequence R55L-R57L mutant of Tat protein
MEPVDPKLEPWKHPGSQPKTACNNCYCKKCCFHCQVCFTKKGLGISYGRKKRRQLRLSPQDSETHQVSLSKQPASQPRGDPTGPKESKKKVERETETDPVD

SEQ ID NO: 14 - protein, 101 aa, Artificial Sequence
  Description of Artificial Sequence R55L-G79A mutant of Tat protein
MEPVDPKLEPWKHPGSQPKTACNNCYCKKCCFHCQVCFTKKGLGISYGRKKRRQLRRSPQDSETHQVSLSKQPASQPRADPTGPKESKKKVERETETDPVD

SEQ ID NO: 15 - protein, 101 aa, Artificial Sequence
  Description of Artificial Sequence R55L-K89L of Tat protein
MEPVDPKLEPWKHPGSQPKTACNNCYCKKCCFHCQVCFTKKGLGISYGRKKRRQLRRSPQDSETHQVSLSKQPASQPRGDPTGPKESKLKVERETETDPVD

SEQ ID NO: 16 - protein, 101 aa, Artificial Sequence
  Description of Artificial Sequence R55L-E92Q of Tat protein
MEPVDPKLEPWKHPGSQPKTACNNCYCKKCCFHCQVCFTKKGLGISYGRKKRRQLRRSPQDSETHQVSLSKQPASQPRGDPTGPKESKKKVQRETETDPVD

SEQ ID NO: 17 - protein, 101 aa, Artificial Sequence
  Description of Artificial Sequence R57L-G79A of Tat protein
MEPVDPKLEPWKHPGSQPKTACNNCYCKKCCFHCQVCFTKKGLGISYGRKKRRQRRLSPQDSETHQVSLSKQPASQPRADPTGPKESKKKVERETETDPVD

SEQ ID NO: 18 - protein, 101 aa, Artificial Sequence
  Description of Artificial Sequence R57L-K89L mutant of Tat protein
MEPVDPKLEPWKHPGSQPKTACNNCYCKKCCFHCQVCFTKKGLGISYGRKKRRQRRLSPQDSETHQVSLSKQPASQPRGDPTGPKESKLKVERETETDPVD

SEQ ID NO: 19 - protein, 101 aa, Artificial Sequence
  Description of Artificial Sequence R57L-E92Q mutant of Tat protein
MEPVDPKLEPWKHPGSQPKTACNNCYCKKCCFHCQVCFTKKGLGISYGRKKRRQRRLSPQDSETHQVSLSKQPASQPRGDPTGPKESKKKVQRETETDPVD

SEQ ID NO: 20 - protein, 101 aa, Artificial Sequence
  Description of Artificial Sequence G79A-K89L mutant of Tat protein
MEPVDPKLEPWKHPGSQPKTACNNCYCKKCCFHCQVCFTKKGLGISYGRKKRRQRRRSPQDSETHQVSLSKQPASQPRADPTGPKESKLKVERETETDPVD

SEQ ID NO: 21 - protein, 101 aa, Artificial Sequence
  Description of Artificial Sequence G79A-E92Q mutant of Tat protein
MEPVDPKLEPWKHPGSQPKTACNNCYCKKCCFHCQVCFTKKGLGISYGRKKRRQRRRSPQDSETHQVSLSKQPASQPRADPTGPKESKKKVQRETETDPVD

SEQ ID NO: 22 - protein, 101 aa, Artificial Sequence
  Description of Artificial Sequence K89L-E92Q mutant of Tat protein
MEPVDPKLEPWKHPGSQPKTACNNCYCKKCCFHCQVCFTKKGLGISYGRKKRRQRRRSPQDSETHQVSLSKQPASQPRGDPTGPKESKLKVQRETETDPVD

SEQ ID NO: 23 - protein, 101 aa, Artificial Sequence
  Description of Artificial Sequence C27S-K51T-R52L mutant of Tat protein
MEPVDPKLEPWKHPGSQPKTACNNCYSKKCCFHCQVCFTKKGLGISYGRKTLRQRRRSPQDSETHQVSLSKQPASQPRGDPTGPKESKKKVERETETDPVD

SEQ ID NO: 24 - protein, 101 aa, Artificial Sequence
  Description of Artificial Sequence C27S-K51T-R55L mutant of Tat protein
MEPVDPKLEPWKHPGSQPKTACNNCYSKKCCFHCQVCFTKKGLGISYGRKTRRQLRRSPQDSETHQVSLSKQPASQPRGDPTGPKESKKKVERETETDPVD

SEQ ID NO: 25 - protein, 101 aa, Artificial Sequence
  Description of Artificial Sequence C27S-K51T-R57L mutant of Tat protein
MEPVDPKLEPWKHPGSQPKTACNNCYSKKCCFHCQVCFTKKGLGISYGRKTRRQRRLSPQDSETHQVSLSKQPASQPRGDPTGPKESKKKVERETETDPVD

SEQ ID NO: 26 - protein, 101 aa, Artificial Sequence
  Description of Artificial Sequence C27S-K51T-G79A mutant of Tat protein
MEPVDPKLEPWKHPGSQPKTACNNCYSKKCCFHCQVCFTKKGLGISYGRKTRRQRRRSPQDSETHQVSLSKQPASQPRADPTGPKESKKKVERETETDPVD

SEQ ID NO: 27 - protein, 101 aa, Artificial Sequence
  Description of Artificial Sequence C27S-K51T-K89L mutant of Tat protein
MEPVDPKLEPWKHPGSQPKTACNNCYSKKCCFHCQVCFTKKGLGISYGRKTRRQRRRSPQDSETHQVSLSKQPASQPRGDPTGPKESKLKVERETETDPVD

SEQ ID NO: 28 - protein, 101 aa, Artificial Sequence
  Description of Artificial Sequence C27S-K51T-E92Q mutant of Tat protein
MEPVDPKLEPWKHPGSQPKTACNNCYSKKCCFHCQVCFTKKGLGISYGRKTRRQRRRSPQDSETHQVSLSKQPASQPRGDPTGPKESKKKVQRETETDPVD

SEQ ID NO: 29 - protein, 101 aa, Artificial Sequence
  Description of Artificial Sequence C27S-R52L-R55L mutant of Tat protein
MEPVDPKLEPWKHPGSQPKTACNNCYSKKCCFHCQVCFTKKGLGISYGRKKLRQLRRSPQDSETHQVSLSKQPASQPRGDPTGPKESKKKVERETETDPVD

SEQ ID NO: 30 - protein, 101 aa, Artificial Sequence
  Description of Artificial Sequence C27S-R52L-R57L mutant of Tat protein
MEPVDPKLEPWKHPGSQPKTACNNCYSKKCCFHCQVCFTKKGLGISYGRKKLRQRRLSPQDSETHQVSLSKQPASQPRGDPTGPKESKKKVERETETDPVD

SEQ ID NO: 31 - protein, 101 aa, Artificial Sequence
  Description of Artificial Sequence C27S-R52L-G79A mutant of Tat protein
MEPVDPKLEPWKHPGSQPKTACNNCYSKKCCFHCQVCFTKKGLGISYGRKKLRQRRRSPQDSETHQVSLSKQPASQPRADPTGPKESKKKVERETETDPVD

SEQ ID NO: 32 - protein, 101 aa, Artificial Sequence
  Description of Artificial Sequence C27S-R52L-K89L mutant of Tat protein
MEPVDPKLEPWKHPGSQPKTACNNCYSKKCCFHCQVCFTKKGLGISYGRKKLRQRRRSPQDSETHQVSLSKQPASQPRGDPTGPKESKLKVERETETDPVD

SEQ ID NO: 33 - protein, 101 aa, Artificial Sequence
  Description of Artificial Sequence C27S-R52L-E92Q mutant of Tat protein
MEPVDPKLEPWKHPGSQPKTACNNCYSKKCCFHCQVCFTKKGLGISYGRKKLRQRRRSPQDSETHQVSLSKQPASQPRGDPTGPKESKKKVQRETETDPVD

SEQ ID NO: 34 - protein, 101 aa, Artificial Sequence
  Description of Artificial Sequence C27S-R55L-R57L mutant of Tat protein
MEPVDPKLEPWKHPGSQPKTACNNCYSKKCCFHCQVCFTKKGLGISYGRKKRRQLRLSPQDSETHQVSLSKQPASQPRGDPTGPKESKKKVERETETDPVD

SEQ ID NO: 35 - protein, 101 aa, Artificial Sequence
  Description of Artificial Sequence C27S-R55L-G79A mutant of Tat protein
MEPVDPKLEPWKHPGSQPKTACNNCYSKKCCFHCQVCFTKKGLGISYGRKKRRQLRRSPQDSETHQVSLSKQPASQPRADPTGPKESKKKVERETETDPVD

SEQ ID NO: 36 - protein, 101 aa, Artificial Sequence
  Description of Artificial Sequence C27S-R55L-K89L mutant of Tat protein
MEPVDPKLEPWKHPGSQPKTACNNCYSKKCCFHCQVCFTKKGLGISYGRKKRRQLRRSPQDSETHQVSLSKQPASQPRGDPTGPKESKLKVERETETDPVD

SEQ ID NO: 37 - protein, 101 aa, Artificial Sequence
  Description of Artificial Sequence C27S-R55L-E92Q mutant of Tat protein
MEPVDPKLEPWKHPGSQPKTACNNCYSKKCCFHCQVCFTKKGLGISYGRKKRRQLRRSPQDSETHQVSLSKQPASQPRGDPTGPKESKKKVQRETETDPVD

SEQ ID NO: 38 - protein, 101 aa, Artificial Sequence
  Description of Artificial Sequence C27S-R57L-G79A mutant of Tat protein
MEPVDPKLEPWKHPGSQPKTACNNCYSKKCCFHCQVCFTKKGLGISYGRKKRRQRRLSPQDSETHQVSLSKQPASQPRADPTGPKESKKKVERETETDPVD

SEQ ID NO: 39 - protein, 101 aa, Artificial Sequence
  Description of Artificial Sequence C27S-R57L-K89L mutant of Tat protein
MEPVDPKLEPWKHPGSQPKTACNNCYSKKCCFHCQVCFTKKGLGISYGRKKRRQRRLSPQDSETHQVSLSKQPASQPRGDPTGPKESKLKVERETETDPVD

SEQ ID NO: 40 - protein, 101 aa, Artificial Sequence
  Description of Artificial Sequence C27S-R57L-E92Q mutant of Tat protein
MEPVDPKLEPWKHPGSQPKTACNNCYSKKCCFHCQVCFTKKGLGISYGRKKRRQRRLSPQDSETHQVSLSKQPASQPRGDPTGPKESKKKVQRETETDPVD

SEQ ID NO: 41 - protein, 101 aa, Artificial Sequence
  Description of Artificial Sequence C27S-G79A-K89L mutant of Tat protein
MEPVDPKLEPWKHPGSQPKTACNNCYSKKCCFHCQVCFTKKGLGISYGRKKRRQRRRSPQDSETHQVSLSKQPASQPRADPTGPKESKLKVERETETDPVD

SEQ ID NO: 42 - protein, 101 aa, Artificial Sequence
  Description of Artificial Sequence C27S-G79A-E92Q mutant of Tat protein
MEPVDPKLEPWKHPGSQPKTACNNCYSKKCCFHCQVCFTKKGLGISYGRKKRRQRRRSPQDSETHQVSLSKQPASQPRADPTGPKESKKKVQRETETDPVD

SEQ ID NO: 43 - protein, 101 aa, Artificial Sequence
  Description of Artificial Sequence C27S-K89L-E92Q mutant of Tat protein
MEPVDPKLEPWKHPGSQPKTACNNCYSKKCCFHCQVCFTKKGLGISYGRKKRRQRRRSPQDSETHQVSLSKQPASQPRGDPTGPKESKLKVQRETETDPVD

SEQ ID NO: 44 - protein, 101 aa, Artificial Sequence
  Description of Artificial Sequence C27S-K51T-R52L-G79A mutant of Tat protein
MEPVDPKLEPWKHPGSQPKTACNNCYSKKCCFHCQVCFTKKGLGISYGRKTLRQRRRSPQDSETHQVSLSKQPASQPRADPTGPKESKKKVERETETDPVD

SEQ ID NO: 45 - protein, 101 aa, Artificial Sequence
  Description of Artificial Sequence C27S-K51T-R52L-K89L mutant of Tat protein
MEPVDPKLEPWKHPGSQPKTACNNCYSKKCCFHCQVCFTKKGLGISYGRKTLRQRRRSPQDSETHQVSLSKQPASQPRGDPTGPKESKLKVERETETDPVD

SEQ ID NO: 46 - protein, 101 aa, Artificial Sequence
  Description of Artificial Sequence C27S-K51T-R52L-E92Q mutant of Tat protein
MEPVDPKLEPWKHPGSQPKTACNNCYSKKCCFHCQVCFTKKGLGISYGRKTLRQRRRSPQDSETHQVSLSKQPASQPRGDPTGPKESKKKVQRETETDPVD

SEQ ID NO: 47 - protein, 101 aa, Artificial Sequence
  Description of Artificial Sequence C27S-K51T-R55L-G79A mutant of Tat protein
MEPVDPKLEPWKHPGSQPKTACNNCYSKKCCFHCQVCFTKKGLGISYGRKTRRQLRRSPQDSETHQVSLSKQPASQPRADPTGPKESKKKVERETETDPVD

SEQ ID NO: 48 - protein, 101 aa, Artificial Sequence
  Description of Artificial Sequence C27S-K51T-R55L-K89L mutant of Tat protein
MEPVDPKLEPWKHPGSQPKTACNNCYSKKCCFHCQVCFTKKGLGISYGRKTRRQLRRSPQDSETHQVSLSKQPASQPRGDPTGPKESKLKVERETETDPVD

SEQ ID NO: 49 - protein, 101 aa, Artificial Sequence
  Description of Artificial Sequence C27S-K51T-R55L-E92Q mutant of Tat protein
MEPVDPKLEPWKHPGSQPKTACNNCYSKKCCFHCQVCFTKKGLGISYGRKTRRQLRRSPQDSETHQVSLSKQPASQPRGDPTGPKESKKKVQRETETDPVD

SEQ ID NO: 50 - protein, 101 aa, Artificial Sequence
  Description of Artificial Sequence C27S-K51T-R57L-G79A mutant of Tat protein
MEPVDPKLEPWKHPGSQPKTACNNCYSKKCCFHCQVCFTKKGLGISYGRKTRRQRRLSPQDSETHQVSLSKQPASQPRADPTGPKESKKKVERETETDPVD

SEQ ID NO: 51 - protein, 101 aa, Artificial Sequence
  Description of Artificial Sequence C27S-K51T-R57L-K89L mutant of Tat protein
MEPVDPKLEPWKHPGSQPKTACNNCYSKKCCFHCQVCFTKKGLGISYGRKTRRQRRLSPQDSETHQVSLSKQPASQPRGDPTGPKESKLKVERETETDPVD

SEQ ID NO: 52 - protein, 101 aa, Artificial Sequence
  Description of Artificial Sequence C27S-K51T-R57L-E92Q mutant of Tat protein
MEPVDPKLEPWKHPGSQPKTACNNCYSKKCCFHCQVCFTKKGLGISYGRKTRRQRRLSPQDSETHQVSLSKQPASQPRGDPTGPKESKKKVQRETETDPVD

SEQ ID NO: 53 - protein, 101 aa, Artificial Sequence
  Description of Artificial Sequence C27S-K51T-G79A-K89L mutant of Tat protein
MEPVDPKLEPWKHPGSQPKTACNNCYSKKCCFHCQVCFTKKGLGISYGRKTRRQRRRSPQDSETHQVSLSKQPASQPRADPTGPKESKLKVERETETDPVD

SEQ ID NO: 54 - protein, 101 aa, Artificial Sequence
  Description of Artificial Sequence C27S-K51T-G79A-E92Q mutant of Tat protein
MEPVDPKLEPWKHPGSQPKTACNNCYSKKCCFHCQVCFTKKGLGISYGRKTRRQRRRSPQDSETHQVSLSKQPASQPRADPTGPKESKKKVQRETETDPVD

SEQ ID NO: 55 - protein, 101 aa, Artificial Sequence
  Description of Artificial Sequence C27S-K51T-K89L-E92Q mutant of Tat protein
MEPVDPKLEPWKHPGSQPKTACNNCYSKKCCFHCQVCFTKKGLGISYGRKTRRQRRRSPQDSETHQVSLSKQPASQPRGDPTGPKESKLKVQRETETDPVD

SEQ ID NO: 56 - protein, 101 aa, Artificial Sequence
  Description of Artificial Sequence C27S-R52L-G79A-K89L mutant of Tat protein
MEPVDPKLEPWKHPGSQPKTACNNCYSKKCCFHCQVCFTKKGLGISYGRKKLRQRRRSPQDSETHQVSLSKQPASQPRADPTGPKESKLKVERETETDPVD

SEQ ID NO: 57 - protein, 101 aa, Artificial Sequence
  Description of Artificial Sequence C27S-R52L-G79A-E92Q mutant of Tat protein
MEPVDPKLEPWKHPGSQPKTACNNCYSKKCCFHCQVCFTKKGLGISYGRKKLRQRRRSPQDSETHQVSLSKQPASQPRADPTGPKESKKKVQRETETDPVD

SEQ ID NO: 58 - protein, 101 aa, Artificial Sequence
  Description of Artificial Sequence C27S-R52L-K89L-E92Q mutant of Tat protein
MEPVDPKLEPWKHPGSQPKTACNNCYSKKCCFHCQVCFTKKGLGISYGRKKLRQRRRSPQDSETHQVSLSKQPASQPRGDPTGPKESKLKVQRETETDPVD

SEQ ID NO: 59 - protein, 101 aa, Artificial Sequence
  Description of Artificial Sequence C27S-R52L-R55L-G79A mutant of Tat protein
MEPVDPKLEPWKHPGSQPKTACNNCYSKKCCFHCQVCFTKKGLGISYGRKKLRQLRRSPQDSETHQVSLSKQPASQPRADPTGPKESKKKVERETETDPVD

SEQ ID NO: 60 - protein, 101 aa, Artificial Sequence
  Description of Artificial Sequence C27S-R52L-R55L-K89Lmutant of Tat protein
MEPVDPKLEPWKHPGSQPKTACNNCYSKKCCFHCQVCFTKKGLGISYGRKKLRQLRRSPQDSETHQVSLSKQPASQPRGDPTGPKESKLKVERETETDPVD

SEQ ID NO: 61 - protein, 101 aa, Artificial Sequence
  Description of Artificial Sequence C27S-R52L-R55L-E92Qmutant of Tat protein
MEPVDPKLEPWKHPGSQPKTACNNCYSKKCCFHCQVCFTKKGLGISYGRKKLRQLRRSPQDSETHQVSLSKQPASQPRGDPTGPKESKKKVQRETETDPVD

SEQ ID NO: 62 - protein, 101 aa, Artificial Sequence
  Description of Artificial Sequence C27S-R52L-R57L-G79Amutant of Tat protein
MEPVDPKLEPWKHPGSQPKTACNNCYSKKCCFHCQVCFTKKGLGISYGRKKLRQRRLSPQDSETHQVSLSKQPASQPRADPTGPKESKKKVERETETDPVD

SEQ ID NO: 63 - protein, 101 aa, Artificial Sequence
  Description of Artificial Sequence C27S-R52L-R57L-K89Lmutant of Tat protein
MEPVDPKLEPWKHPGSQPKTACNNCYSKKCCFHCQVCFTKKGLGISYGRKKLRQRRLSPQDSETHQVSLSKQPASQPRGDPTGPKESKLKVERETETDPVD

SEQ ID NO: 64 - protein, 101 aa, Artificial Sequence
  Description of Artificial Sequence C27S-R52L-R57L-E92Qmutant of Tat protein
MEPVDPKLEPWKHPGSQPKTACNNCYSKKCCFHCQVCFTKKGLGISYGRKKLRQRRLSPQDSETHQVSLSKQPASQPRGDPTGPKESKKKVQRETETDPVD

SEQ ID NO: 65 - protein, 101 aa, Artificial Sequence
  Description of Artificial Sequence C27S-R55L-G79A-K89Lmutant of Tat protein
MEPVDPKLEPWKHPGSQPKTACNNCYSKKCCFHCQVCFTKKGLGISYGRKKRRQLRRSPQDSETHQVSLSKQPASQPRADPTGPKESKLKVERETETDPVD

SEQ ID NO: 66 - protein, 101 aa, Artificial Sequence
  Description of Artificial Sequence C27S-R55L-G79A-E92Qmutant of Tat protein
MEPVDPKLEPWKHPGSQPKTACNNCYSKKCCFHCQVCFTKKGLGISYGRKKRRQLRRSPQDSETHQVSLSKQPASQPRADPTGPKESKKKVQRETETDPVD

SEQ ID NO: 67 - protein, 101 aa, Artificial Sequence
  Description of Artificial Sequence C27S-R55L-K89L-E92Qmutant of Tat protein
MEPVDPKLEPWKHPGSQPKTACNNCYSKKCCFHCQVCFTKKGLGISYGRKKRRQLRRSPQDSETHQVSLSKQPASQPRGDPTGPKESKLKVQRETETDPVD

SEQ ID NO: 68 - protein, 101 aa, Artificial Sequence
  Description of Artificial Sequence C27S-R55L-R57L-G79Amutant of Tat protein
MEPVDPKLEPWKHPGSQPKTACNNCYSKKCCFHCQVCFTKKGLGISYGRKKRRQLRLSPQDSETHQVSLSKQPASQPRADPTGPKESKKKVERETETDPVD

SEQ ID NO: 69 - protein, 101 aa, Artificial Sequence
  Description of Artificial Sequence C27S-R55L-R57L-K89Lmutant of Tat protein
MEPVDPKLEPWKHPGSQPKTACNNCYSKKCCFHCQVCFTKKGLGISYGRKKRRQLRLSPQDSETHQVSLSKQPASQPRGDPTGPKESKLKVERETETDPVD

SEQ ID NO: 70 - protein, 101 aa, Artificial Sequence
  Description of Artificial Sequence C27S-R55L-R57L-E92Qmutant of Tat protein
MEPVDPKLEPWKHPGSQPKTACNNCYSKKCCFHCQVCFTKKGLGISYGRKKRRQLRLSPQDSETHQVSLSKQPASQPRGDPTGPKESKKKVQRETETDPVD

SEQ ID NO: 71 - protein, 101 aa, Artificial Sequence
  Description of Artificial Sequence C27S-R57L-G79A-K89Lmutant of Tat protein
MEPVDPKLEPWKHPGSQPKTACNNCYSKKCCFHCQVCFTKKGLGISYGRKKRRQRRLSPQDSETHQVSLSKQPASQPRADPTGPKESKLKVERETETDPVD

SEQ ID NO: 72 - protein, 101 aa, Artificial Sequence
  Description of Artificial Sequence C27S-R57L-G79A-E92Qmutant of Tat protein
MEPVDPKLEPWKHPGSQPKTACNNCYSKKCCFHCQVCFTKKGLGISYGRKKRRQRRLSPQDSETHQVSLSKQPASQPRADPTGPKESKKKVQRETETDPVD

SEQ ID NO: 73 - protein, 101 aa, Artificial Sequence
  Description of Artificial Sequence C27S-R57L-K89L-E92Qmutant of Tat protein
MEPVDPKLEPWKHPGSQPKTACNNCYSKKCCFHCQVCFTKKGLGISYGRKKRRQRRLSPQDSETHQVSLSKQPASQPRGDPTGPKESKLKVQRETETDPVD

SEQ ID NO: 74 - protein, 101 aa, Artificial Sequence
  Description of Artificial Sequence C27S-G79A-K89L-E92Qmutant of Tat protein
MEPVDPKLEPWKHPGSQPKTACNNCYSKKCCFHCQVCFTKKGLGISYGRKKRRQRRRSPQDSETHQVSLSKQPASQPRADPTGPKESKLKVQRETETDPVD

SEQ ID NO: 75 - protein, 101 aa, Artificial Sequence
  Description of Artificial Sequence C27S-K51T-G79A-K89L-E92Q mutant of Tat protein
MEPVDPKLEPWKHPGSQPKTACNNCYSKKCCFHCQVCFTKKGLGISYGRKTRRQRRRSPQDSETHQVSLSKQPASQPRADPTGPKESKLKVQRETETDPVD

SEQ ID NO: 76 - protein, 101 aa, Artificial Sequence
  Description of Artificial Sequence C27S-K51T-R52L-R55L-G79A mutant of Tat protein
MEPVDPKLEPWKHPGSQPKTACNNCYSKKCCFHCQVCFTKKGLGISYGRKTLRQLRRSPQDSETHQVSLSKQPASQPRADPTGPKESKKKVERETETDPVD

SEQ ID NO: 77 - protein, 101 aa, Artificial Sequence
  Description of Artificial Sequence C27S-K51T-R52L-R55L-K89L mutant of Tat protein
MEPVDPKLEPWKHPGSQPKTACNNCYSKKCCFHCQVCFTKKGLGISYGRKTLRQLRRSPQDSETHQVSLSKQPASQPRGDPTGPKESKLKVERETETDPVD

SEQ ID NO: 78 - protein, 101 aa, Artificial Sequence
  Description of Artificial Sequence C27S-K51T-R52L-R55L-E92Q mutant of Tat protein
MEPVDPKLEPWKHPGSQPKTACNNCYSKKCCFHCQVCFTKKGLGISYGRKTLRQLRRSPQDSETHQVSLSKQPASQPRGDPTGPKESKKKVQRETETDPVD

SEQ ID NO: 79 - protein, 101 aa, Artificial Sequence
  Description of Artificial Sequence C27S-K51T-R52L-R57L-G79A mutant of Tat protein
MEPVDPKLEPWKHPGSQPKTACNNCYSKKCCFHCQVCFTKKGLGISYGRKTLRQRRLSPQDSETHQVSLSKQPASQPRADPTGPKESKKKVERETETDPVD

SEQ ID NO: 80 - protein, 101 aa, Artificial Sequence
  Description of Artificial Sequence C27S-K51T-R52L-R57L-K89L mutant of Tat protein
MEPVDPKLEPWKHPGSQPKTACNNCYSKKCCFHCQVCFTKKGLGISYGRKTLRQRRLSPQDSETHQVSLSKQPASQPRGDPTGPKESKLKVERETETDPVD

SEQ ID NO: 81 - protein, 101 aa, Artificial Sequence
  Description of Artificial Sequence C27S-K51T-R52L-R57L-E92Q mutant of Tat protein
MEPVDPKLEPWKHPGSQPKTACNNCYSKKCCFHCQVCFTKKGLGISYGRKTLRQRRLSPQDSETHQVSLSKQPASQPRGDPTGPKESKKKVQRETETDPVD

SEQ ID NO: 82 - protein, 101 aa, Artificial Sequence
  Description of Artificial Sequence C27S-K51T-R52L-G79A-K89L mutant of Tat protein
MEPVDPKLEPWKHPGSQPKTACNNCYSKKCCFHCQVCFTKKGLGISYGRKTLRQRRRSPQDSETHQVSLSKQPASQPRADPTGPKESKLKVERETETDPVD

SEQ ID NO: 83 - protein, 101 aa, Artificial Sequence
  Description of Artificial Sequence C27S-K51T-R52L-G79A-E92Q mutant of Tat protein
MEPVDPKLEPWKHPGSQPKTACNNCYSKKCCFHCQVCFTKKGLGISYGRKTLRQRRRSPQDSETHQVSLSKQPASQPRADPTGPKESKKKVQRETETDPVD

SEQ ID NO: 84 - protein, 101 aa, Artificial Sequence
  Description of Artificial Sequence C27S-K51T-R52L-K89L-E92Q mutant of Tat protein
MEPVDPKLEPWKHPGSQPKTACNNCYSKKCCFHCQVCFTKKGLGISYGRKTLRQRRRSPQDSETHQVSLSKQPASQPRGDPTGPKESKLKVQRETETDPVD

SEQ ID NO: 85 - protein, 101 aa, Artificial Sequence
  Description of Artificial Sequence C27S-K51T-R55L-R57L-G79A mutant of Tat protein
MEPVDPKLEPWKHPGSQPKTACNNCYSKKCCFHCQVCFTKKGLGISYGRKTRRQLRLSPQDSETHQVSLSKQPASQPRADPTGPKESKKKVERETETDPVD

SEQ ID NO: 86 - protein, 101 aa, Artificial Sequence
  Description of Artificial Sequence C27S-K51T-R55L-R57L-K89L mutant of Tat protein
MEPVDPKLEPWKHPGSQPKTACNNCYSKKCCFHCQVCFTKKGLGISYGRKTRRQLRLSPQDSETHQVSLSKQPASQPRGDPTGPKESKLKVERETETDPVD

SEQ ID NO: 87 - protein, 101 aa, Artificial Sequence
  Description of Artificial Sequence C27S-K51T-R55L-R57L-E92Q mutant of Tat protein
MEPVDPKLEPWKHPGSQPKTACNNCYSKKCCFHCQVCFTKKGLGISYGRKTRRQLRLSPQDSETHQVSLSKQPASQPRGDPTGPKESKKKVQRETETDPVD

SEQ ID NO: 88 - protein, 101 aa, Artificial Sequence
  Description of Artificial Sequence C27S-K51T-R55L-G79A-K89L mutant of Tat protein
MEPVDPKLEPWKHPGSQPKTACNNCYSKKCCFHCQVCFTKKGLGISYGRKTRRQLRRSPQDSETHQVSLSKQPASQPRADPTGPKESKLKVERETETDPVD

SEQ ID NO: 89 - protein, 101 aa, Artificial Sequence
  Description of Artificial Sequence C27S-K51T-R55L-G79A-E92Q mutant of Tat protein
MEPVDPKLEPWKHPGSQPKTACNNCYSKKCCFHCQVCFTKKGLGISYGRKTRRQLRRSPQDSETHQVSLSKQPASQPRADPTGPKESKKKVQRETETDPVD

SEQ ID NO: 90 - protein, 101 aa, Artificial Sequence
  Description of Artificial Sequence C27S-K51T-R55L-K89L-E92Q mutant of Tat protein
MEPVDPKLEPWKHPGSQPKTACNNCYSKKCCFHCQVCFTKKGLGISYGRKTRRQLRRSPQDSETHQVSLSKQPASQPRGDPTGPKESKLKVQRETETDPVD

SEQ ID NO: 91 - protein, 101 aa, Artificial Sequence
  Description of Artificial Sequence C27S-K51T-R57L-G79A-K89L mutant of Tat protein
MEPVDPKLEPWKHPGSQPKTACNNCYSKKCCFHCQVCFTKKGLGISYGRKTRRQRRLSPQDSETHQVSLSKQPASQPRADPTGPKESKLKVERETETDPVD

SEQ ID NO: 92 - protein, 101 aa, Artificial Sequence
  Description of Artificial Sequence C27S-K51T-R57L-G79A-E92Q mutant of Tat protein
MEPVDPKLEPWKHPGSQPKTACNNCYSKKCCFHCQVCFTKKGLGISYGRKTRRQRRLSPQDSETHQVSLSKQPASQPRADPTGPKESKKKVQRETETDPVD

SEQ ID NO: 93 - protein, 101 aa, Artificial Sequence
  Description of Artificial Sequence C27S-K51T-R57L-K89L-E92Q mutant of Tat protein
MEPVDPKLEPWKHPGSQPKTACNNCYSKKCCFHCQVCFTKKGLGISYGRKTRRQRRLSPQDSETHQVSLSKQPASQPRGDPTGPKESKLKVQRETETDPVD

SEQ ID NO: 94 - protein, 101 aa, Artificial Sequence
  Description of Artificial Sequence C27S-R52L-R55L-R57L-G79A mutant of Tat protein
MEPVDPKLEPWKHPGSQPKTACNNCYSKKCCFHCQVCFTKKGLGISYGRKKLRQLRLSPQDSETHQVSLSKQPASQPRADPTGPKESKKKVERETETDPVD

SEQ ID NO: 95 - protein, 101 aa, Artificial Sequence
  Description of Artificial Sequence C27S-R52L-R55L-R57L-K89L mutant of Tat protein
MEPVDPKLEPWKHPGSQPKTACNNCYSKKCCFHCQVCFTKKGLGISYGRKKLRQLRLSPQDSETHQVSLSKQPASQPRGDPTGPKESKLKVERETETDPVD

SEQ ID NO: 96 - protein, 101 aa, Artificial Sequence
  Description of Artificial Sequence C27S-R52L-R55L-R57L-E92Q mutant of Tat protein
MEPVDPKLEPWKHPGSQPKTACNNCYSKKCCFHCQVCFTKKGLGISYGRKKLRQLRLSPQDSETHQVSLSKQPASQPRGDPTGPKESKKKVQRETETDPVD

SEQ ID NO: 97 - protein, 101 aa, Artificial Sequence
  Description of Artificial Sequence C27S-R52L-R55L-G79A-K89L mutant of Tat protein
MEPVDPKLEPWKHPGSQPKTACNNCYSKKCCFHCQVCFTKKGLGISYGRKKLRQLRRSPQDSETHQVSLSKQPASQPRADPTGPKESKLKVERETETDPVD

SEQ ID NO: 98 - protein, 101 aa, Artificial Sequence
  Description of Artificial Sequence C27S-R52L-R55L-G79A-E92Q mutant of Tat protein
MEPVDPKLEPWKHPGSQPKTACNNCYSKKCCFHCQVCFTKKGLGISYGRKKLRQLRRSPQDSETHQVSLSKQPASQPRADPTGPKESKKKVQRETETDPVD

SEQ ID NO: 99 - protein, 101 aa, Artificial Sequence
  Description of Artificial Sequence C27S-R52L-R55L-K89L-E92Q mutant of Tat protein
MEPVDPKLEPWKHPGSQPKTACNNCYSKKCCFHCQVCFTKKGLGISYGRKKLRQLRRSPQDSETHQVSLSKQPASQPRGDPTGPKESKLKVQRETETDPVD

SEQ ID NO: 100 - protein, 101 aa, Artificial Sequence
  Description of Artificial Sequence C27S-R52L-R57L-G79A-K89L mutant of Tat protein
MEPVDPKLEPWKHPGSQPKTACNNCYSKKCCFHCQVCFTKKGLGISYGRKKLRQRRLSPQDSETHQVSLSKQPASQPRADPTGPKESKLKVERETETDPVD

SEQ ID NO: 101 - protein, 101 aa, Artificial Sequence
  Description of Artificial Sequence C27S-R52L-R57L-G79A-E92Q mutant of Tat protein
MEPVDPKLEPWKHPGSQPKTACNNCYSKKCCFHCQVCFTKKGLGISYGRKKLRQRRLSPQDSETHQVSLSKQPASQPRADPTGPKESKKKVQRETETDPVD

SEQ ID NO: 102 - protein, 101 aa, Artificial Sequence
  Description of Artificial Sequence C27S-R52L-R57L-K89L-E92Q mutant of Tat protein
MEPVDPKLEPWKHPGSQPKTACNNCYSKKCCFHCQVCFTKKGLGISYGRKKLRQRRLSPQDSETHQVSLSKQPASQPRGDPTGPKESKLKVQRETETDPVD

SEQ ID NO: 103 - protein, 101 aa, Artificial Sequence
  Description of Artificial Sequence C27S-R52L-G79A-K89L-E92Q mutant of Tat protein
MEPVDPKLEPWKHPGSQPKTACNNCYSKKCCFHCQVCFTKKGLGISYGRKKLRQRRRSPQDSETHQVSLSKQPASQPRADPTGPKESKLKVQRETETDPVD

SEQ ID NO: 104 - protein, 101 aa, Artificial Sequence
  Description of Artificial Sequence C27S-R55L-R57L-G79A-K89L mutant of Tat protein
MEPVDPKLEPWKHPGSQPKTACNNCYSKKCCFHCQVCFTKKGLGISYGRKKRRQLRLSPQDSETHQVSLSKQPASQPRADPTGPKESKLKVERETETDPVD

SEQ ID NO: 105 - protein, 101 aa, Artificial Sequence
  Description of Artificial Sequence C27S-R55L-R57L-G79A-E92Q mutant of Tat protein
MEPVDPKLEPWKHPGSQPKTACNNCYSKKCCFHCQVCFTKKGLGISYGRKKRRQLRLSPQDSETHQVSLSKQPASQPRADPTGPKESKKKVQRETETDPVD

SEQ ID NO: 106 - protein, 101 aa, Artificial Sequence
  Description of Artificial Sequence C27S-R55L-R57L-K89L-E92Q mutant of Tat protein
MEPVDPKLEPWKHPGSQPKTACNNCYSKKCCFHCQVCFTKKGLGISYGRKKRRQLRLSPQDSETHQVSLSKQPASQPRGDPTGPKESKLKVQRETETDPVD

SEQ ID NO: 107 - protein, 101 aa, Artificial Sequence
  Description of Artificial Sequence C27S-R55L-G79A-K89L-E92Q mutant of Tat protein
MEPVDPKLEPWKHPGSQPKTACNNCYSKKCCFHCQVCFTKKGLGISYGRKKRRQLRRSPQDSETHQVSLSKQPASQPRADPTGPKESKLKVQRETETDPVD

SEQ ID NO: 108 - protein, 101 aa, Artificial Sequence
  Description of Artificial Sequence C27S-R57L-G79A-K89L-E92Q mutant of Tat protein
MEPVDPKLEPWKHPGSQPKTACNNCYSKKCCFHCQVCFTKKGLGISYGRKKRRQRRLSPQDSETHQVSLSKQPASQPRADPTGPKESKLKVQRETETDPVD

SEQ ID NO: 109 - protein, 6 aa, Human immunodeficiency virus
RKGLGI

SEQ ID NO: 110 - protein, 9 aa, Human immunodeficiency virus
RKKRRQRRR

SEQ ID NO: 111 - protein, 15 aa, Human immunodeficiency virus
EPVDPKLEPWKHPGS

SEQ ID NO: 112 - DNA, 29 bp, Artificial Sequence
  Description of Artificial Sequence Synthetic primer
ggcaggaagcttagacagctgcgaagatc

SEQ ID NO: 113 - DNA, 32 bp, Artificial Sequence
  Description of Artificial Sequence Synthetic primer
gatcttcgcagctgtctaagcttcttcctgcc

SEQ ID NO: 114 - DNA, 22 bp, Artificial Sequence
  Description of Artificial Sequence Synthetic primer
gaattcatggagccagtagatc

SEQ ID NO: 115 - DNA, 21 bp, Artificial Sequence
  Description of Artificial Sequence Synthetic primer
agatctctaatcgaccggatc

SEQ ID NO: 116 - DNA, 26 bp, Artificial Sequence
  Description of Artificial Sequence Synthetic primer
aaagaattcatggagccagtagatcc

SEQ ID NO: 117 - DNA, 35 bp, Artificial Sequence
  Description of Artificial Sequence Synthetic primer
aaagatctctaatcgaccggatctgtctctgtctc

SEQ ID NO: 118 - DNA, 35 bp, Artificial Sequence
  Description of Artificial Sequence Synthetic primer
aagtcgacctaatcgaccggatctgtctctgtctc

SEQ ID NO: 119 - DNA, 37 bp, Artificial Sequence
  Description of Artificial Sequence Synthetic primer
ccagtagatcctaaactagagcccttcaagcatccag

SEQ ID NO: 120 - DNA, 22 bp, Artificial Sequence
  Description of Artificial Sequence Synthetic primer
acaattgctattcgaaaaagtg

SEQ ID NO: 121 - DNA, 22 bp, Artificial Sequence
  Description of Artificial Sequence Synthetic primer
cactttttcgaatagcaattgt

SEQ ID NO: 122 - DNA, 21 bp, Artificial Sequence
  Description of Artificial Sequence Synthetic primer
atctcatatggcaggcggaag

SEQ ID NO: 123 - DNA, 21 bp, Artificial Sequence
  Description of Artificial Sequence Synthetic primer
cttccgcctgccatatgagat

SEQ ID NO: 124 - DNA, 22 bp, Artificial Sequence
  Description of Artificial Sequence Synthetic primer
ggcaggaagacccggagacagc

SEQ ID NO: 125 - DNA, 22 bp, Artificial Sequence
  Description of Artificial Sequence Synthetic primer
gctgtctccgggtcttcctgcc

SEQ ID NO: 126 - DNA, 32 bp, Artificial Sequence
  Description of Artificial Sequence Synthetic primer
ggcaggaagaagcttagacagcgacgaagatc

SEQ ID NO: 127 - DNA, 32 bp, Artificial Sequence
  Description of Artificial Sequence Synthetic primer
gatcttcgtcgctgtctaagcttcttcctgcc

SEQ ID NO: 128 - DNA, 32 bp, Artificial Sequence
  Description of Artificial Sequence Synthetic primer
ggcaggaagaagcggagacagctgcgaagatc

SEQ ID NO: 129 - DNA, 32 bp, Artificial Sequence
  Description of Artificial Sequence Synthetic primer
gatcttcgcagctgtctccgcttcttcctgcc

SEQ ID NO: 130 - DNA, 26 bp, Artificial Sequence
  Description of Artificial Sequence Synthetic primer
gacagcgacgactatctcctcaagac

SEQ ID NO: 131 - DNA, 26 bp, Artificial Sequence
  Description of Artificial Sequence Synthetic primer
gtcttgaggagatagtcgtcgctgtc

SEQ ID NO: 132 - DNA, 23 bp, Artificial Sequence
  Description of Artificial Sequence Synthetic primer
cagccccgagcggatccgacagg

SEQ ID NO: 133 - DNA, 23 bp, Artificial Sequence
  Description of Artificial Sequence Synthetic primer
cctgtcggatccgctcggggctg

SEQ ID NO: 134 - DNA, 38 bp, Artificial Sequence
  Description of Artificial Sequence Synthetic primer
ctgtctctgtctctctctccaccttaagcttcgattcc

SEQ ID NO: 135 - DNA, 38 bp, Artificial Sequence
  Description of Artificial Sequence Synthetic primer
ctgtctctgtctctctttgcaccttcttcttcgaatcc

SEQ ID NO: 136 - DNA, 32 bp, Artificial Sequence
  Description of Artificial Sequence Synthetic primer
ggcaggaagaagcttagacagctgcgaagatc

SEQ ID NO: 137 - DNA, 32 bp, Artificial Sequence; identical to SEQ ID NO: 113
  Description of Artificial Sequence Synthetic primer

SEQ ID NO: 138 - DNA, 34 bp, Artificial Sequence
  Description of Artificial Sequence Synthetic primer
gaagcggagacagctgcgactatctcctcaagac

SEQ ID NO: 139 - DNA, 34 bp, Artificial Sequence
  Description of Artificial Sequence Synthetic primer
gtcttgaggagatagtcgcagctgtctccgcttc

SEQ ID NO: 140 - DNA, 29 bp, Artificial Sequence
  Description of Artificial Sequence Synthetic primer
gacagcgacgaagagcacctcaagacagt

SEQ ID NO: 141 - DNA, 29 bp, Artificial Sequence
  Description of Artificial Sequence Synthetic primer
actgtcttgaggtgctcttcgtcgctgtc

(19) United States
(12) Patent Application Publication
(10) Pub. No.: US 2005/0164164 A1
(43) Pub. Date: Jul. 28, 2005

(54) Hiv-1 virus tat-protein mutants

(76) Inventors: Christophe Guillon, Bron (FR); Aurelie Chedal-Bornu, Mets-Tessy (FR); Bernard Mandrand, Villeurbanne (FR)

Correspondence Address: YOUNG & THOMPSON, 745 SOUTH 23RD STREET, 2ND FLOOR, ARLINGTON, VA, 22202, US

(21) Appl. No.: 10/501,223

(22) PCT Filed: Jan. 9, 2003
(86) PCT No.: PCT/FR03/00051

(30) Foreign Application Priority Data
Jan. 11, 2002 (FR) 0200319

(51) Int. Cl. C12Q 1/70; C07K 14/16

(52) U.S. Cl. 435/5; 530/350; 530/388.3

(57) ABSTRACT

The invention relates to the use of a protein mutation method for preparing detoxified and immunogenic mutants of the wild-type HIV-1 virus Tat-protein. The invention also relates to a method for preparing detoxified and immunogenic mutants of the wild-type HIV-1 virus Tat-protein, comprising a first step in which wild-type Tat-protein mutants are prepared, a second step in which detoxified mutants having no transcellular activity but an altered nuclear localization are screened, and a third step in which immunogenic mutants capable of inducing antibodies directed against both mutants and the wild Tat protein are screened. Steps two and three are reversible.

A subject of the present invention is HIV-1 virus Tat-protein mutants and a pharmaceutical composition, in particular a vaccine, comprising at least one of said mutants.

The HIV virus is the etiological agent of AIDS. The HIV belongs to the family of human retroviruses (Retroviridae) and to the sub-family of the lentiviruses. Among the two types of HIV (HIV-1 and HIV-2), HIV-1 is the more cytopathic and the more prevalent world-wide, in particular in Western countries. HIV-1 infection is accompanied by early dysfunction of the immune system in humans infected by the virus.

Like the other retroviruses, HIV-1 has genes which code for structural proteins of the virus. The gag gene codes for the protein which forms the virion core, including the p24 antigen. The pol gene codes for the enzymes responsible for reverse transcription (reverse transcriptase) and integration (integrase). The env gene codes for the envelope glycoproteins. However HIV-1 is more complex than the other retroviruses and contains six other genes (tat, rev, nef, vif, vpr and vpu) which code for proteins involved in regulation of the expression of the genes of the virus. The genome of HIV-1 also comprises the 5′ and 3′ LTRs (Long Terminal Repeats) which include regulation elements involved in the expression of the genes of the virus.

In vivo, Tat is a protein necessary for the replication of HIV-1. The function(s) of Tat in transcription have been much studied and it is now pretty clear that one of Tat's main roles is the regulation of the transcription from the 5′ LTR. Tat is an activator of transcription by transactivation of the 5′ LTR, via its fixation to the TAR sequence at the same time as to other cell factors, resulting in an increase in viral transcription and elongation. The transactivation of the LTR by the Tat protein is essential both for the expression of the genes and the replication of the virus. The transactivation of the viral promoter by the Tat protein (17, 18) allows the large-scale production of viral messenger RNAs the transfer of which into the cytoplasm depends on another regulatory protein, the Rev protein. Tat and Rev regulate the expression of HIV-1 (7). The Tat protein is secreted by cells infected by HIV-1. Once outside the cell, it is capable of being internalized by neighbouring cells (9, 14), infected or not, thus being able to induce modifications to the state of activation of non-infected T lymphocytes. It is therefore directly involved in the progression of AIDS and probably in pathologies associated with AIDS, such as Kaposi's sarcoma.

The complete Tat protein is made up of 101 amino acids, residues 1-72 being coded by a first exon and residues 73-101 being coded by a second exon. The Tat protein is strongly conserved. A truncated form of 86 amino acids, which does not correspond to the native form, exists in a few laboratory strains obtained after culture passages. This truncated form is due to the introduction of a stop codon at position 87 during the culture passages, but more than 90% of the Tat proteins studied maintain the configuration of 101 amino acids. Although amino acids 87-101 could not contribute greatly to ex vivo propagation, their conservation in the natural isolates of HIV-1 which replicate is an indication of their biological importance. HIV-1's native Tat protein of 101 amino acids is made up of five physical domains, but the molecular mechanism by which it acts has not yet been completely explained. Briefly, these five domains are described in the publication of Jeang, K. T et al. (18). In this publication, domain 1 corresponds to amino acids 1-20 which are rich in acid residues, domain 2 corresponds to amino acids 21-40 which are rich in cysteine residues (7 cysteine residues, 6 of which are very strongly conserved), domain 3 corresponds to amino acids 41-48 and contains the RKGLGI motif common to HIV-1, HIV-2 and SIV, domain 4 corresponds to amino acids 49-72 and contains a basic RKKRRQRRR motif and domain 5 corresponds to amino acids 73-101 and comprises an RGD motif. The role of domain 1 has not yet been explained. It has only been shown that changes in a single amino acid in this domain were well tolerated and did not alter the functionality of the Tat protein. One hypothesis put forward is that domain 1 could be involved in transactivation. Changing six cysteines out of the seven in domain 2 suppresses the functionality of the Tat protein. This domain is important for transactivation. The role of domain 3 has not been elucidated. Domain 4 confers the properties of Tat fixation on the TAR RNA and is important for nuclear localization as well as for transcellular transport of the Tat protein. Domain 5 would also be involved in the transcellular transport of the Tat protein.

In the detailed description of the invention which follows, the present inventors started from the sequence of the ACH320.2A.2.1 strain (NCBI accession no. U34604) and refined the notion of domains given in the publication of Jeang, K. T et al. (18). Thus and with reference to this particular strain, in the present invention domain 1 corresponds to amino acids 1-21 (role not elucidated), domain 2 corresponds to amino acids 22-37 (involved in transactivation), domain 3 corresponds to amino acids 38-48 (role unknown) and domain 5 corresponds to amino acids 73-101 (transcellular transport). In domain 4 corresponding to amino acids 49-72, it is the peptide 49-57 which is important for fixation to the TAR RNA, for nuclear localization and for the transcellular transport of Tat (18).

The development of a vaccine against HIV-1 is awaited world-wide. In patients infected with HIV-1, an immune response to Tat and Rev is detected only in individuals for whom the infection does not progress to AIDS. (26). Several studies of vaccination using Tat and/or Rev in the SIV animal model have shown a partial or complete protection against infection (4-6, 21). However, direct transposition of these vaccination protocols to humans is not possible. It has been shown inter alia that Tat has toxic effects in vitro (19, 22). These toxic effects include (i) deregulation of cell signals involved in apoptosis (28, 30), (ii) deregulation of the expression of parts of genes of the immune system such as the gene coding for interleukin-2 (29), or genes coding for the molecules of the major histocompatibility complex (MHC) of class I (16), and/or (iii) induction of angiogenesis (1, 2, 20). The Tat protein must therefore be detoxified before being used as a vaccine antigen. One team chose to detoxify the Tat protein by chemical inactivation (10). However, such inactivation can be carried out only with a view to using recombinant proteins as vaccine antigens. In order to be able to use the Tat protein in the form of nucleic acid in a recombinant vector, living or not, only a genetic detoxification can be envisaged. The present inventors therefore chose to explore this route for the detoxification of the Tat protein, by directed mutagenesis in order to allow its use both as a vaccine protein sub-unit and/or as part of a vaccination vector.

The present invention relates to the use of a protein mutation process for the preparation of detoxified and immunogenic wild-type Tat-protein mutants.

By wild-type Tat protein “mutants” is meant mutants obtained by substitution or replacement of one or more amino acids.

By “detoxified wild-type Tat-protein mutants” is meant a Tat protein no longer having the following toxic effects:
when it is secreted by an infected cell, the Tat protein is toxic in exogenous form to cells not infected with HIV-1, due to its capacity to induce a cell signalization by fixation to surface receptors, and its ability to be internalized by non-infected cells and transported to the nucleus of the target cell;
in exogenous and endogenous manner, the Tat protein will be localized in the nucleus of the target cell and induce the regulation of the expression of cell genes, being able to involve the transactivating properties or domain 5 of the Tat protein.

By “immunogenic wild-type Tat-protein mutants” is meant a mutant capable of inducing the production of antibodies after injection into a model animal, these antibodies having the capacity to react both with the Tat-protein mutant but also with the wild-type Tat protein.

The invention also relates to a process for preparing detoxified and immunogenic wild-type Tat-protein mutants characterized in that it comprises:
a stage of preparation of wild-type Tat-protein mutants, in particular by mutation of the nucleic acid coding for the wild-type Tat protein,
a stage of screening the detoxified mutants characterized by an absence of transcellular activity and an alteration of the nuclear localization, and optionally by an absence of transactivating activity, and
a stage of screening the immunogenic mutants characterized by their ability to induce antibodies directed against both said mutants and the wild-type Tat protein, the order of the last two stages being reversible.

The absence of transcellular activity also means an absence of transcellular transport and can be detected in a cell line established by the absence of activation of the viral promotor of the LTR-reporter gene construction, for example that of chloramphenicol acetyltransferase (CAT), the expression of which depends on the viral promoter (LTR) in an established cell line (31), after these cells are brought into contact with a Tat-protein mutant produced in exogenous manner, for example by a cell line other than that containing the reporter gene dependent on the viral LTR (32).

The alteration of the nuclear localization can be defined as the presence of the Tat protein in the cytoplasmic compartment of cells transfected by nucleic acids coding for the wild-type Tat-protein mutants, for example in the 72 hours following transfection, and can be detected either by optical microscopy after transfection of cell lines by the nucleic acids coding for the Tat-protein mutants by techniques involving immunomarking of the product of these genes, or by the detection after transfection of the product of the translation of nucleic acids containing the gene coding for a Tat mutant fused to the gene coding for an autofluorescent protein such as the EGFP protein (33, 34).

The absence of transactivating activity corresponds to the absence of activation of the viral promoter and can be detected by the absence of expression of a reporter gene, for example that of chloramphenicol acetyltransferase (CAT), the expression of which depends on the viral promoter (LTR) in an established cell line, after transfection of this line by nucleic acids coding for the wild-type Tat-protein mutants (31).

The invention also relates to a detoxified and immunogenic mutant of the Tat protein of the HIV-1 virus, characterized in that it comprises at least two mutations in regions 4 and/or 5 of the wild-type Tat protein, and in that, when the mutation is in the region of domain 4, it is in the part delimited by the amino acid in position 49 to the amino acid in position 57, and in that, when the mutation is in domain 5, it is either in the RGD motif, or in the region 88-92, preferably in positions 89 and/or 92, the mutations being mutations by substitution of one amino acid by another.

An advantageous mutant according to the present invention is a mutant as defined above, characterized in that it comprises at least one mutation in region 4.

The invention also relates to a mutant as defined above, characterized in that the mutations in domains 4 and/or 5 are capable of conferring at least one of the following properties:
the cancellation of the transcellular effect of the wild-type Tat protein,
the alteration of the nuclear localization of the wild-type Tat protein.

The invention relates to a mutant as defined above, characterized in that it comprises an additional mutation capable of conferring a loss of the transactivating activity of the wild-type Tat protein.

A Tat protein usable as vaccination antigen will therefore have to meet most of the following criteria:
cancellation of the transcellular effect of Tat (domain 4 and/or 5)
alteration of the nuclear localization of Tat (domain 4)
loss of the transactivating activity (domain 2)
maintenance of the protein's antigenicity (a maximum of 4 or 5 mutations, modifying the CTL epitopes as little as possible)

According to an advantageous embodiment, the present invention relates to a mutant as defined above, characterized in that it comprises a mutation in the N-terminal region of domain 4 of the wild-type Tat protein, in particular in the part delimited by the amino acid in position 49 to the amino acid in position 57.

An advantageous mutant according to the invention is a mutant as defined above, characterized in that it comprises a mutation in the N-terminal region of domain 4 of the wild-type Tat protein in the part delimited by the amino acid in position 49 to the amino acid in position 55.

An advantageous mutant according to the invention is a mutant as defined above, characterized in that it comprises a mutation in at least one of the following regions in domain 5 of the wild-type Tat protein:
the RGD motif,
the region 88-92, preferably in positions 89 and/or 92.

An advantageous mutant according to the invention is a mutant as defined above, characterized in that it comprises a mutation in domain 2 of the wild-type Tat protein, in particular the replacement of any one of the cysteines, advantageously by a serine.

The invention also relates to a mutant as defined above, characterized in that it comprises at least one of the following mutations:
replacement in position 27 of a cysteine by a serine,
replacement in position 51 of a lysine by a threonine,
replacement in position 52 of an arginine by a leucine,
replacement in position 55 of an arginine by a leucine,
replacement in position 57 of an arginine by a leucine,
replacement in position 79 of a glycine by an alanine,
replacement in position 89 of a lysine by a leucine,
replacement in position 92 of a glutamic acid by a glutamine.

The invention also relates to a mutant as defined above, characterized in that it is chosen from the mutants having two mutations as indicated hereafter, each of the mutations being represented by a triplet: letter-figure-letter, the figure of which indicates the position of the mutated amino acid, the letter preceding the figure corresponds to the amino acid to which the mutation relates and the letter following the figure corresponds to the amino acid replacing the amino acid preceding the figure:

| | |
|---|---|
| K51T-R52L | (SEQ ID NO: 2) |
| K51T-R55L | (SEQ ID NO: 3) |
| K51T-R57L | (SEQ ID NO: 4) |
| K51T-G79A | (SEQ ID NO: 5) |
| K51T-K89L | (SEQ ID NO: 6) |
| K51T-E92Q | (SEQ ID NO: 7) |
| R52L-R55L | (SEQ ID NO: 8) |
| R52L-R57L | (SEQ ID NO: 9) |
| R52L-G79A | (SEQ ID NO: 10) |
| R52L-K89L | (SEQ ID NO: 11) |
| R52L-E92Q | (SEQ ID NO: 12) |
| R55L-R57L | (SEQ ID NO: 13) |
| R55L-G79A | (SEQ ID NO: 14) |
| R55L-K89L | (SEQ ID NO: 15) |
| R55L-E92Q | (SEQ ID NO: 16) |
| R57L-G79A | (SEQ ID NO: 17) |
| R57L-K89L | (SEQ ID NO: 18) |
| R57L-E92Q | (SEQ ID NO: 19) |
| G79A-K89L | (SEQ ID NO: 20) |
| G79A-E92Q | (SEQ ID NO: 21) |
| K89L-E92Q | (SEQ ID NO: 22) |

An advantageous mutant according to the present invention is a mutant as defined above, characterized in that it is chosen from the following mutants:

| | |
|---|---|
| K51T-R55L | (SEQ ID NO: 3) |
| R52L-R55L | (SEQ ID NO: 8) |
| R52L-G79A | (SEQ ID NO: 10) |
| R55L-R57L | (SEQ ID NO: 13) |
| G79A-K89L | (SEQ ID NO: 20) |

The invention also relates to a mutant as defined above, characterized in that it is chosen from the mutants having three mutations as indicated hereafter, each of the mutations being represented by a triplet: letter-figure-letter, the figure of which indicates the position of the mutated amino acid, the letter preceding the figure corresponds to the amino acid to which the mutation relates and the letter following the figure corresponds to the amino acid replacing the amino acid preceding the figure:

| | |
|---|---|
| C27S-K51T-R52L | (SEQ ID NO: 23) |
| C27S-K51T-R55L | (SEQ ID NO: 24) |
| C27S-K51T-R57L | (SEQ ID NO: 25) |
| C27S-K51T-G79A | (SEQ ID NO: 26) |
| C27S-K51T-K89L | (SEQ ID NO: 27) |
| C27S-K51T-E92Q | (SEQ ID NO: 28) |
| C27S-R52L-R55L | (SEQ ID NO: 29) |
| C27S-R52L-R57L | (SEQ ID NO: 30) |
| C27S-R52L-G79A | (SEQ ID NO: 31) |
| C27S-R52L-K89L | (SEQ ID NO: 32) |
| C27S-R52L-E92Q | (SEQ ID NO: 33) |
| C27S-R55L-R57L | (SEQ ID NO: 34) |
| C27S-R55L-G79A | (SEQ ID NO: 35) |
| C27S-R55L-K89L | (SEQ ID NO: 36) |
| C27S-R55L-E92Q | (SEQ ID NO: 37) |
| C27S-R57L-G79A | (SEQ ID NO: 38) |
| C27S-R57L-K89L | (SEQ ID NO: 39) |
| C27S-R57L-E92Q | (SEQ ID NO: 40) |
| C27S-G79A-K89L | (SEQ ID NO: 41) |
| C27S-G79A-E92Q | (SEQ ID NO: 42) |
| C27S-K89L-E92Q | (SEQ ID NO: 43) |

The present invention also relates to a mutant as defined above, characterized in that it is chosen from the following mutants:

| | |
|---|---|
| C27S-K51T-R55L | (SEQ ID NO: 24) |
| C27S-R52L-R55L | (SEQ ID NO: 29) |
| C27S-R52L-G79A | (SEQ ID NO: 31) |

The present invention relates to a mutant as defined above, characterized in that it is chosen from the mutants having four mutations as indicated hereafter, each of the mutations being represented by a triplet: letter-figure-letter, the figure of which indicates the position of the mutated amino acid, the letter preceding the figure corresponds to the amino acid to which the mutation relates and the letter following the figure corresponds to the amino acid replacing the amino acid preceding the figure:

| | |
|---|---|
| C27S-K51T-R52L-G79A | (SEQ ID NO: 44) |
| C27S-K51T-R52L-K89L | (SEQ ID NO: 45) |
| C27S-K51T-R52L-E92Q | (SEQ ID NO: 46) |
| C27S-K51T-R55L-G79A | (SEQ ID NO: 47) |
| C27S-K51T-R55L-K89L | (SEQ ID NO: 48) |
| C27S-K51T-R55L-E92Q | (SEQ ID NO: 49) |
| C27S-K51T-R57L-G79A | (SEQ ID NO: 50) |
| C27S-K51T-R57L-K89L | (SEQ ID NO: 51) |
| C27S-K51T-R57L-E92Q | (SEQ ID NO: 52) |
| C27S-K51T-G79A-K89L | (SEQ ID NO: 53) |
| C27S-K51T-G79A-E92Q | (SEQ ID NO: 54) |
| C27S-K51T-K89L-E92Q | (SEQ ID NO: 55) |
| C27S-R52L-G79A-K89L | (SEQ ID NO: 56) |
| C27S-R52L-G79A-E92Q | (SEQ ID NO: 57) |
| C27S-R52L-K89L-E92Q | (SEQ ID NO: 58) |
| C27S-R52L-R55L-G79A | (SEQ ID NO: 59) |
| C27S-R52L-R55L-K89L | (SEQ ID NO: 60) |
| C27S-R52L-R55L-E92Q | (SEQ ID NO: 61) |
| C27S-R52L-R57L-G79A | (SEQ ID NO: 62) |
| C27S-R52L-R57L-K89L | (SEQ ID NO: 63) |
| C27S-R52L-R57L-E92Q | (SEQ ID NO: 64) |
| C27S-R55L-G79A-K89L | (SEQ ID NO: 65) |
| C27S-R55L-G79A-E92Q | (SEQ ID NO: 66) |
| C27S-R55L-K89L-E92Q | (SEQ ID NO: 67) |
| C27S-R55L-R57L-G79A | (SEQ ID NO: 68) |
| C27S-R55L-R57L-K89L | (SEQ ID NO: 69) |
| C27S-R55L-R57L-E92Q | (SEQ ID NO: 70) |
| C27S-R57L-G79A-K89L | (SEQ ID NO: 71) |
| C27S-R57L-G79A-E92Q | (SEQ ID NO: 72) |
| C27S-R57L-K89L-E92Q | (SEQ ID NO: 73) |
| C27S-G79A-K89L-E92Q | (SEQ ID NO: 74) |

An advantageous mutant according to the present invention is characterized in that it is chosen from the following mutants:

| | |
|---|---|
| C27S-K51T-R55L-G79A | (SEQ ID NO: 47) |
| C27S-K51T-R55L-K89L | (SEQ ID NO: 48) |
| C27S-K51T-R55L-E92Q | (SEQ ID NO: 49) |
| C27S-R52L-R55L-G79A | (SEQ ID NO: 59) |

The present invention relates to a mutant as defined above, characterized in that it is chosen from the mutants having five mutations as indicated hereafter, each of the mutations being represented by a triplet: letter-figure-letter, the figure of which indicates the position of the mutated amino acid, the letter preceding the figure corresponds to the amino acid to which the mutation relates and the letter following the figure corresponds to the amino acid replacing the amino acid preceding the figure:

| | |
|---|---|
| C27S-K51T-G79A-K89L-E92Q | (SEQ ID NO: 75) |
| C27S-K51T-R52L-R55L-G79A | (SEQ ID NO: 76) |
| C27S-K51T-R52L-R55L-K89L | (SEQ ID NO: 77) |
| C27S-K51T-R52L-R55L-E92Q | (SEQ ID NO: 78) |
| C27S-K51T-R52L-R57L-G79A | (SEQ ID NO: 79) |
| C27S-K51T-R52L-R57L-K89L | (SEQ ID NO: 80) |
| C27S-K51T-R52L-R57L-E92Q | (SEQ ID NO: 81) |
| C27S-K51T-R52L-G79A-K89L | (SEQ ID NO: 82) |
| C27S-K51T-R52L-G79A-E92Q | (SEQ ID NO: 83) |
| C27S-K51T-R52L-K89L-E92Q | (SEQ ID NO: 84) |
| C27S-K51T-R55L-R57L-G79A | (SEQ ID NO: 85) |
| C27S-K51T-R55L-R57L-K89L | (SEQ ID NO: 86) |
| C27S-K51T-R55L-R57L-E92Q | (SEQ ID NO: 87) |
| C27S-K51T-R55L-G79A-K89L | (SEQ ID NO: 88) |
| C27S-K51T-R55L-G79A-E92Q | (SEQ ID NO: 89) |
| C27S-K51T-R55L-K89L-E92Q | (SEQ ID NO: 90) |
| C27S-K51T-R57L-G79A-K89L | (SEQ ID NO: 91) |
| C27S-K51T-R57L-G79A-E92Q | (SEQ ID NO: 92) |
| C27S-K51T-R57L-K89L-E92Q | (SEQ ID NO: 93) |
| C27S-R52L-R55L-R57L-G79A | (SEQ ID NO: 94) |
| C27S-R52L-R55L-R57L-K89L | (SEQ ID NO: 95) |
| C27S-R52L-R55L-R57L-E92Q | (SEQ ID NO: 96) |
| C27S-R52L-R55L-G79A-K89L | (SEQ ID NO: 97) |
| C27S-R52L-R55L-G79A-E92Q | (SEQ ID NO: 98) |
| C27S-R52L-R55L-K89L-E92Q | (SEQ ID NO: 99) |
| C27S-R52L-R57L-G79A-K89L | (SEQ ID NO: 100) |
| C27S-R52L-R57L-G79A-E92Q | (SEQ ID NO: 101) |
| C27S-R52L-R57L-K89L-E92Q | (SEQ ID NO: 102) |
| C27S-R52L-G79A-K89L-E92Q | (SEQ ID NO: 103) |
| C27S-R55L-R57L-G79A-K89L | (SEQ ID NO: 104) |
| C27S-R55L-R57L-G79A-E92Q | (SEQ ID NO: 105) |
| C27S-R55L-R57L-K89L-E92Q | (SEQ ID NO: 106) |
| C27S-R55L-G79A-K89L-E92Q | (SEQ ID NO: 107) |
| C27S-R57L-G79A-K89L-E92Q | (SEQ ID NO: 108) |

An advantageous mutant according to the invention is a mutant as defined above, characterized in that it is chosen from the following mutants:

| | |
|---|---|
| C27S-K51T-R55L-G79A-K89L | (SEQ ID NO: 88) |
| C27S-K51T-R55L-G79A-E92Q | (SEQ ID NO: 89) |

The present invention also relates to nucleotide sequences coding for one of the mutants as defined above.

The present invention also relates to the cell lines transfected with a nucleotide sequence of the invention.

The invention also relates to antibodies directed against one of the mutants as defined above, and not recognizing domain D1 of the wild-type protein.

Such antibodies are selected by testing and eliminating those which possess an affinity for a peptide corresponding to domain D1 of Tat, containing at least the sequence EPVDPKLEPWKHPGS (residues 2-16), for example in an Elisa format test.

The antibodies according to the invention do or do not recognize the wild-type protein.

An advantageous class of antibodies according to the invention comprises the antibodies as defined above, recognizing the wild-type protein.

The antibodies according to the invention are polyclonal or monoclonal antibodies.

The abovementioned polyclonal antibodies are obtained by immunization of an animal with at least one mutant according to the invention, followed by recovery of the sought antibodies in purified form, by taking a sample of the serum of said animal, and separation of said antibodies from the other constituents of the serum, in particular by affinity chromatography over a column on which is fixed an antigen specifically recognized by the antibodies, in particular a mutant according to the invention.

The monoclonal antibodies according to the invention can be obtained by the hybridomas technique the general principle of which is recalled hereafter.

In a first phase, an animal, generally a mouse (or cells in culture within the framework of immunizations in vitro), is immunized with a mutant according to the invention, the B lymphocytes of which are then capable of producing antibodies to said mutant. These antibody-producing lymphocytes are then fused with “immortal” myelomatous cells (murine in the example) in order to produce hybridomas. From the thus-obtained heterogeneous mixture of cells, a selection is then made of cells capable of producing a particular antibody and multiplying indefinitely. Each hybridoma is multiplied in clone form, each leading to the production of a monoclonal antibody the recognition properties of which vis-a-vis the mutant of the invention can be tested for example by ELISA, by immunotransfer in one or two dimensions, by immunofluorescence, or using a biocaptor. The monoclonal antibodies thus selected are subsequently purified in particular according to the affinity chromatography technique described above.

The present invention also relates to a pharmaceutical composition, in particular a vaccine, containing as active ingredient at least one of the mutants as defined above or at least one of the nucleotide sequences as defined above, placed under the control of elements necessary to a constitutive expression of one of the mutants as defined above or at least one of the antibodies as defined above, in combination with a pharmaceutically suitable vehicle.

Of course, a person skilled in the art will easily determine the quantity of mutant to be used as a function of the constituents of the pharmaceutical composition.

The present invention also relates to a diagnostic composition for the detection and/or quantification of the HIV-1 virus comprising at least one mutant as defined above, or at least one antibody as defined above.

Of course, a person skilled in the art will easily determine the quantity of mutant to be used as a function of the diagnostic technique used.

The invention also relates to a process for the detection and/or quantification of the HIV-1 virus in a biological sample taken from an individual capable of being infected with HIV-1, such as plasma, serum or tissue, characterized in that it comprises stages consisting of:
bringing said biological sample into contact with a diagnostic composition comprising a mutant as defined above or an antibody as defined above, under predetermined conditions which allow, if necessary, the formation of antibody/antigen complexes between the mutant defined above and antibodies directed against the wild-type Tat protein or between the antibodies defined above and the wild-type Tat protein, and
detecting and/or quantifying the formation of said complexes by any appropriate means.

The processes of detection and/or of quantification of the virus are implemented using standard techniques well known to a person skilled in the art and there can be mentioned, by way of illustration, blots, so-called sandwich techniques and competition techniques.

The invention also relates to the use of at least one mutant as defined above or at least one antibody as defined above for the in vitro diagnosis of the HIV-1 virus in a biological specimen or sample.

The invention also relates to the use of at least one mutant as defined above or at least one antibody as defined above for the preparation of a vaccine composition.

The inventors thus showed that for the abovementioned uses it was necessary to carry out at least one mutation at domain 4 and/or at least one mutation at domain 5 of the Tat protein. They obtained, by directed mutagenesis, Tat-protein mutants which were then selected according to their properties. The mutants retained are chosen from the mutants having at least one of the following mutations: K51T (replacement in position 51 of a lysine by a threonine at domain 4), R52L (replacement of an arginine by a leucine in position 52 in domain 4), R55L (replacement of an arginine by a leucine in position 55 in domain 4), R57L (replacement of an arginine by a leucine in position 57 in domain 4), G79A (replacement of a glycine by an alanine in position 79 in domain 5), K89L (replacement of a lysine by a leucine in position 89 in domain 5) and E92Q (replacement of a glutamic acid by a glutamine in position 92 in domain 5). All the amino acid positions described above and subsequently are given with reference to the complete sequence of 101 amino acids of the strain ACH320.2A.2.1. A subject of the invention is the abovementioned mutants. But the invention also relates to mutants having two mutations at domain 4 of the Tat protein. These “double” mutants are selected from the mutants K51T-R55L, R52L-R55L, R52L-G79A, R55L-R57L and G79A-K89L. The inventors then showed that by combining these double mutations in domain 4 with an additional C27S mutation in domain 2 (replacement of a cysteine by a serine), they obtained very satisfactory results. Thus, the invention also covers a mutant chosen from the “triple” mutants C27S-K51T-R55L, C27S-R52L-R55L and C27S-R52L-G79A. Preferably, the mutant chosen is the mutant C27S-K51T-R55L. Finally, they proved that a “quadruple” mutant combining at least one mutation in domain 2, two mutations in domain 4 and at least one mutation in domain 5 had excellent performances for obtaining a non-toxic Tat protein. The “quadruple” mutant is chosen from the mutants C27S-K51T-R55L-G79A, C27S-K51T-R55L-K89L, C27S-K51T-R55L-E92Q and C27S-R52L-R55L-G79A. Preferably, the mutant chosen is the mutant C27S-K51T-R55L-G79A.

### DESCRIPTION OF FIGURES

FIG. 1 represents a diagrammatic representation of the PCR-directed point mutagenesis technique.

FIG. 2 represents the alignment of the protein sequence of the Tat protein of the strain ACH320.2A.2.1 (SEQ ID NO: 1) and of the protein sequences of the mutated Tat proteins of the invention.

FIGS. 3a and 3b correspond to a diagram representing the transactivating capacity of ACH320.2A.2.1 or its mutants. The results represented for each construction correspond to the average of two independent experiments. In FIG. 3a, the NT (non-transfected) column represents the basal activity of the LTR-CAT construction.

The y-axis represents the transactivation multiplication factor.

FIGS. 4a and 4b represent the intracellular localization of the ACH320.2A.2.1 construction or its mutants.

FIG. 5 represents the transduction capacity of the ACH320.2A.2.1 construction or its mutants. The values indicated correspond to the average of two independent experiments. The cleavage line was determined by calculating the average +3 SD (standard deviation) of the transactivation percentage measured for pEGFP.

The white columns correspond to a 293T/HL3T1 co-culture and the black columns to the transfection of the HL3T1 cells.

The y-axis corresponds to the transactivation percentage relative to wild-type ACH320.2A.2.1.

FIG. 6 represents an additional method for screening the cell lines expressing the mutants of the invention (described in Example 5).

Table 1 represents the oligonucleotides used for the PCR-directed mutagenesis of Tat.

### EXAMPLE 1

### Construction of the Mutated DNA Coding for the Mutated Tat Protein

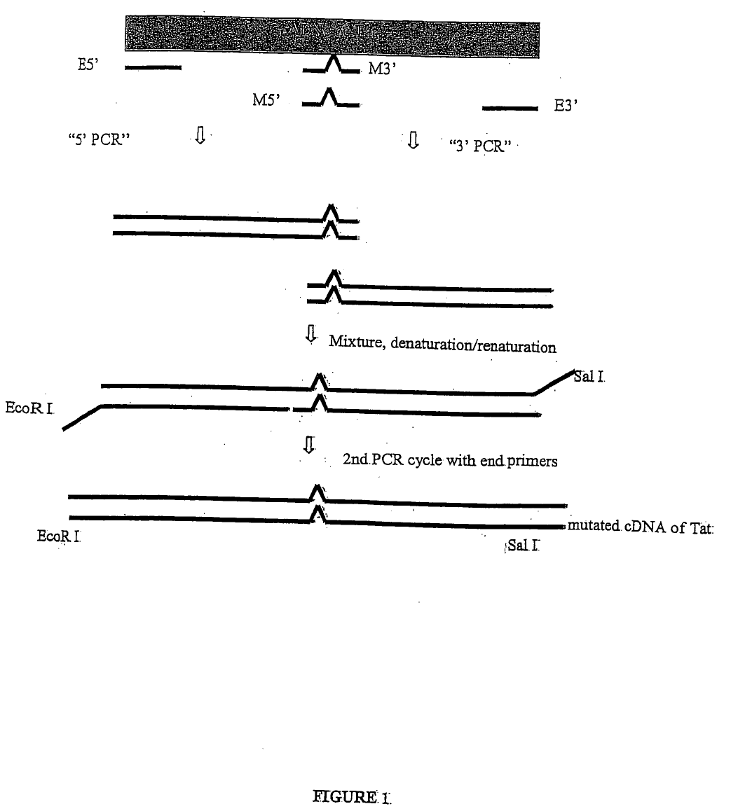

A cDNA fragment comprising 306 base pairs corresponding to the two exons of the wild-type Tat gene of the isolate ACH320.2A.2.1 of HIV-1 (11, 12) is mutated using a commercial PCR kit (Clontech) and the nucleotide primers described in Table 1. The principle of the PCR-directed point mutagenesis is described in FIG. 1.

As shown in FIG. 1, starting from the cDNA of the wild-type tat gene, two PCRs are carried out independently with a primer situated at the end (E5′ or E3′) and an internal primer situated in the gene and carrying the desired mutation (M3′ and M5′, respectively) (first PCR cycle). The two PCR products are then mixed in equimolar manner and a second PCR cycle is carried out with end primers containing the restriction sites for EcoRI at 5′ and SaII at 3′. Thus the cDNAs are obtained mutated at the desired point.

This principle was used for all the mutants except for the K89L and E92Q. mutations For the latter the nucleotides having to be mutated were localized in the proximity of one of the ends of the cDNA, allowing a direct semi-nested PCR-directed mutagenesis using for the first PCR cycle respectively the following pairs of primers: E5′/K89L (M3′) and E5′/E92Q (M5′).

The double mutant R52L-R55L was generated using the following primers for single mutagenesis containing the two mutations:

R52L-R55L (M5′)
5′-GGCAGGAAGCTTAGACAGCTGCGAAGATC-3′
R52L-R55L (M3′)
5′-GATCTTCGCAGCTGTCTAAGCTTCTTCCTGCC-3′

The double mutant R52L-G79A was obtained using as matrix the cDNA of the mutant G79A and as a primer pair the pair R52L (M5′) /R52L (M3′) for the PCR-directed mutagenesis.

The double mutant G79A-K89L was generated by semi-nested PCR using as matrix the cDNA of the mutant G79A and the primer pair E5′/K89L (M3′) for the first PCR cycle.

The triple mutant C27S-K51T-R55L (STL) was obtained using the cDNA of the double mutant K51T-R55L as matrix and a primer pair C27S (M5′)/C27S (M3′) for the PCR-directed mutagenesis.

The triple mutant C27S-R52L-G79A was obtained using as matrix the cDNA of the mutant R52L-G79A and as primer pair the pair C27S (M5′)/C27S (M3′) for the PCR-directed mutagenesis.

The quadruple mutant C27S-K51T-R55L-G79A (STLA) was generated using the cDNA of STL as matrix and the primers G79A (M5′)/G79A (M3′) for the mutagenesis.

All the first PCR cycles were carried out using 0.5 μg of plasmid, 0.29 ng/ml of each of the primers under the following conditions: 1×94° C. 5′ 1×[94° C. 2′50° C. 2′ 72° C. 4′] 25×[94° C. 1′ 50° C. 1′ 72° C. 4′] 1×72° C. 5′.

The primer pair EcoRI/SalI was used for the second cycle of all the PCR-directed mutageneses, except for the mutants R52L, R55L and the double mutant R52L-R55L, for which the primer SalI/3′ was replaced by the primer E6854. In all cases the second cycle was carried out using the same conditions as for the first cycle and 0.5 μl of the products of PCR 5′ and 3′ of the first cycles (FIG. 1). A strip of 323 base pairs was created and was then bound in the pCR2,1-Topo (Invitrogen, K4500-40) plasmid according to the manufacturer's protocol in order to create the pCR-TEX constructions.

The positive clones were then selected after automated sequencing using the DyeTerminator (trade name) sequencing mix on the 377X automatic sequencer (Applied Biosystems). Analysis of the sequences was carried out using the MacVector 7.0 software (Oxford Molecular). Among all the constructions sequenced, all contain only the desired mutation, except one of the clones derived from the product PCR-RS55L, which showed an additional mutation K→T at position 51. This double mutant K51T-R55L was therefore conserved for additional analysis, and the single mutant K51T was generated using the primers K51T (M5′) and K51T (M3′).

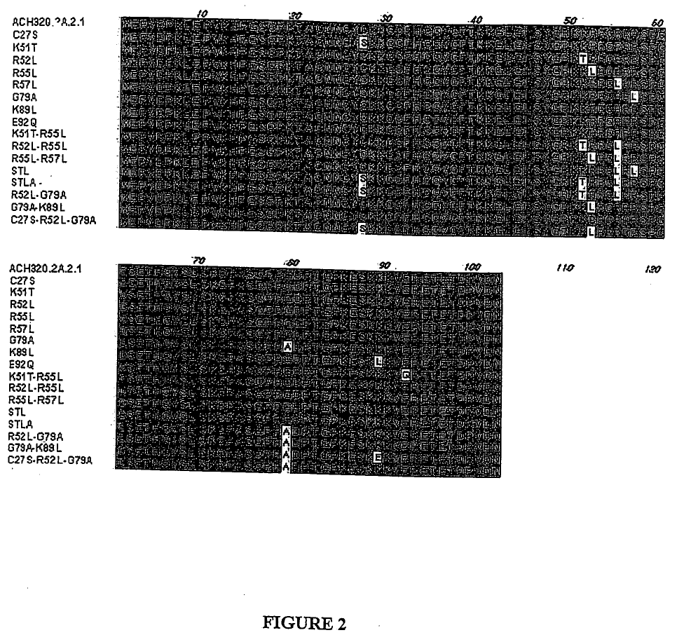

The EcoRI-SalI or EcoRI-EcoRI fragments of the pCR-TEX constructions were sub-cloned in a eukaryotic vector pEGFP-C2 (Clontech) in which the Tat mutant is fused in the C-terminal position with EGFP (Enhanced Fluorescent Green Protein). It was previously shown that the fusion of the EGFP to Tat does not alter Tat's transactivating capacity, nor its cellular localization (25). The cloning stages were carried out in Escherichia coli (E. Coli) DH5α according to standard molecular biology techniques (23). The pEGFP-TEX constructions, in which the expression of the fusion protein is under the control of the Cytomegalovirus (CMV) promoter, were obtained and screened by automatic sequencing for positive clones. The amino acid sequences of the positive clones which were selected are represented in FIG. 2. The DNA of these clones was amplified, purified using the Nucleobond AX kit (trade name) (Macherey-Nagel), according to the manufacturer's instructions.

### EXAMPLE 2

### Transactivating Capacity of the Tat Mutants

In order to study the transactivating capacity of the EGFP-Tat fusion proteins, the cell line HL3T1 was used. This line is a HeLa cell derivative, transfected in stable manner with a chloramphenicol acetyltransferase (CAT) gene dependent on the viral HIV-1 promoter (LTR) (8). One day after seeding of 2.5×10⁵ HL3T1 cells in 6-well plates, the cells were transfected with 2 μg of the pEGFP-TEX constructions using the Exgen 500 kit (marketed by Euromedex) according to the protocol recommended by the manufacturer. After 48 hours to 72 hours of culture, the cells were trypsinized and the quantity of transfected cells was estimated by fluorescence microscopy. The equivalent of 1.5×10³ fluorescent cells was lysed in 100 μl of Tris 0.01M-EDTA 1 nM-NaCl 150 mM (TEN) and subjected to a freezing/thawing stage before treatment for 20 minutes at 65° C. The CAT activity was then measured in a phase-extraction test, as previously described (24). Briefly, 70 μl of cell lysates were incubated for 2 hours at 37° C. with 130 μl of the CAT reaction mixture (Tris-HCl pH=7.5 150 mM, EDTA 0.2 nM, NaCl 30 mM, butyryl coenzyme A 0.3 mg/ml, glycerol 3%, D-threo-[dichloroacetyl-1-¹⁴C]chloramphenicol 0.08 μCi.). The reaction mixture was then extracted using 400 μl of a mixture of pristane (2,6,10,14-tetramethylpentadecane) and xylene in a volume/volume ratio of 2:1. The radioactivity was measured on 300 μl of the resulting organic phase using a scintillation counter.

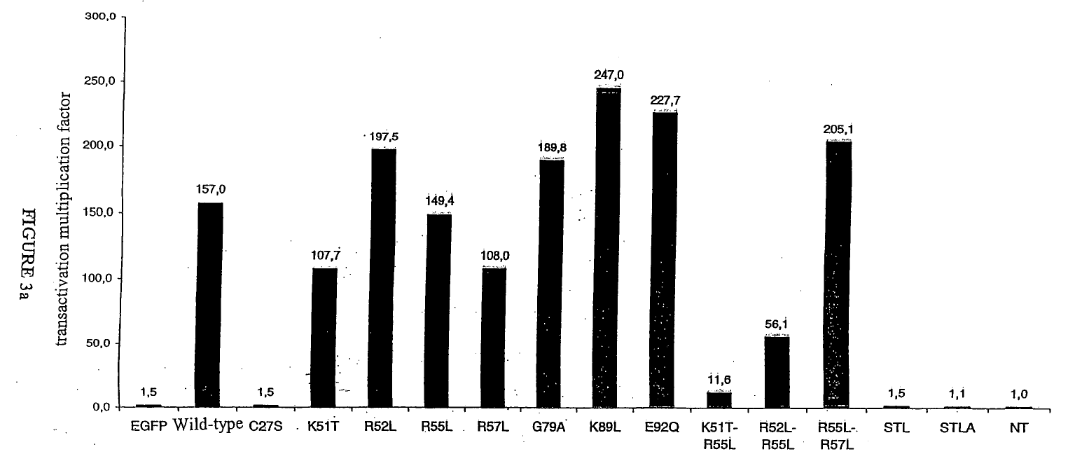

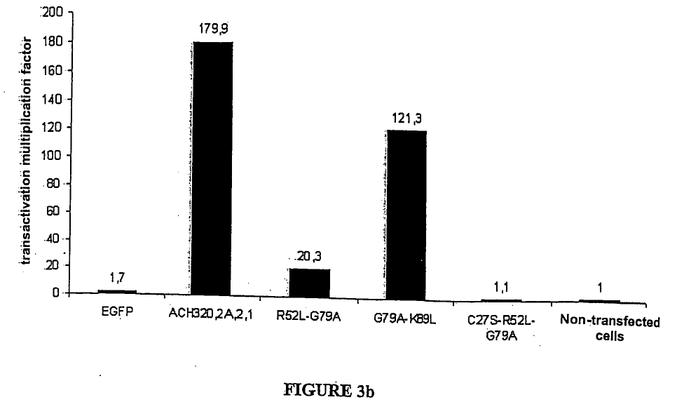

FIGS. 3a and 3b show that none of the single mutations alone allows the complete destruction of the transactivating activity of the Tat protein of ACH320.2A.2.1, except for the mutant C27S. The mutant R55L does not very significantly modify the transactivating activity of ACH320.2A.2.1 Tat. But this mutation combined with one of the mutations K51T or R52L shows a very significant inhibition of the transactivating activity of Tat. This inhibition is total for the triple and quadruple mutants STL and STLA.

### EXAMPLE 3

### Intracellular Localization of the Tat-EGFP Fusion Proteins

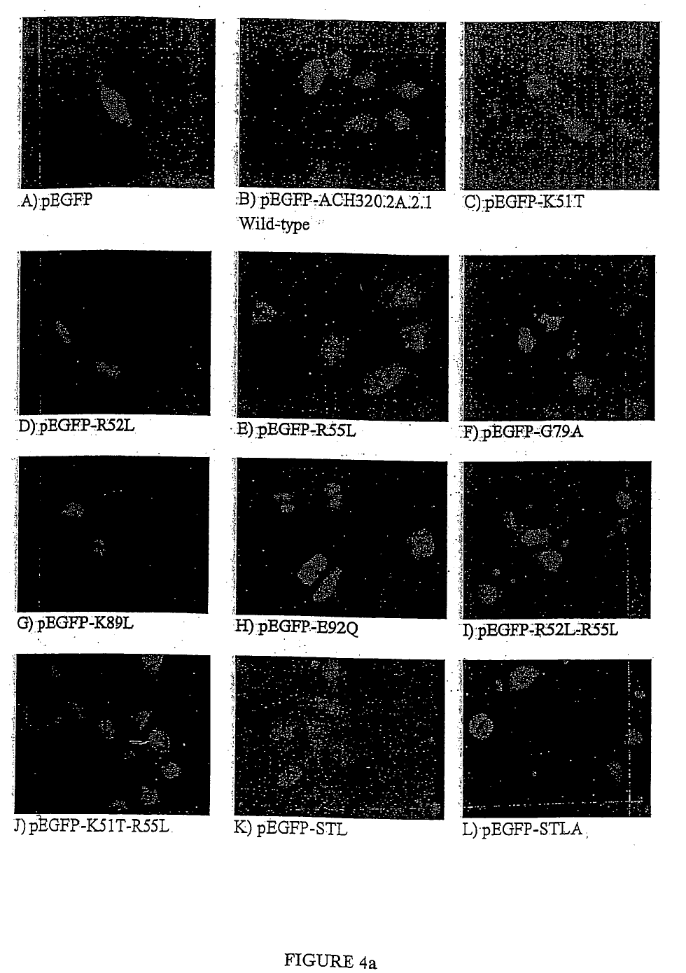

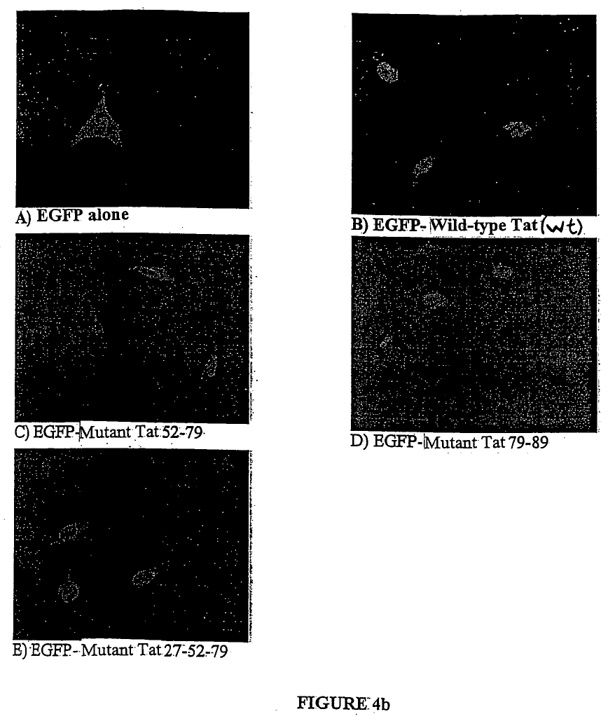

The basic Tat domain is responsible for the nuclear localization of Tat. Certain of the mutations which were constructed affect this basic domain. Also, the inventors wanted to identify the mutations which affected the intracellular localization of Tat. After seeding of 2.5×10⁵ HL3T1 cells on microscope slides, the cells were transfected with 2 μg of each pEGFP-TEX construction using the Exgen 500 kit (marketed by Euromedex) according to the protocol recommended by the manufacturer. After 1, 2 or 3 days the slides are recovered and fixed with 4% paraformaldehyde before observation using an Axioplan 2 (trade name) fluorescence microscope (Zeiss). As shown in FIG. 4a or b (A and B) and as already described (25), the wild-type Tat-EGFP fusion protein showed a nuclear localization after 3 days of culture. The single Tat mutations did not affect this localization (FIG. 4a, C to H), nor the double mutation R52L-R55L (FIG. 4a, I), R52L-G79A (FIG. 4b, C), G79A-K89L (FIG. 4b, D), nor the triple mutant C27S-R52L-G79A (FIG. 4b, E). However, the K51T-R55L combination or multiple mutants containing it (STL, STLA) showed both a nuclear and cytoplasmic localization of the Tat protein-EGFP on the 3rd day (FIG. 4a, J to L), whereas the signal was strictly nuclear on the 1st and 2nd day following the transfection. It therefore seems that Tat's nuclear localization signal is discontinuous and contains at least the residues K51 and R55, but not R52.

### EXAMPLE 4

### Transcellular Activity of the Tat Mutants

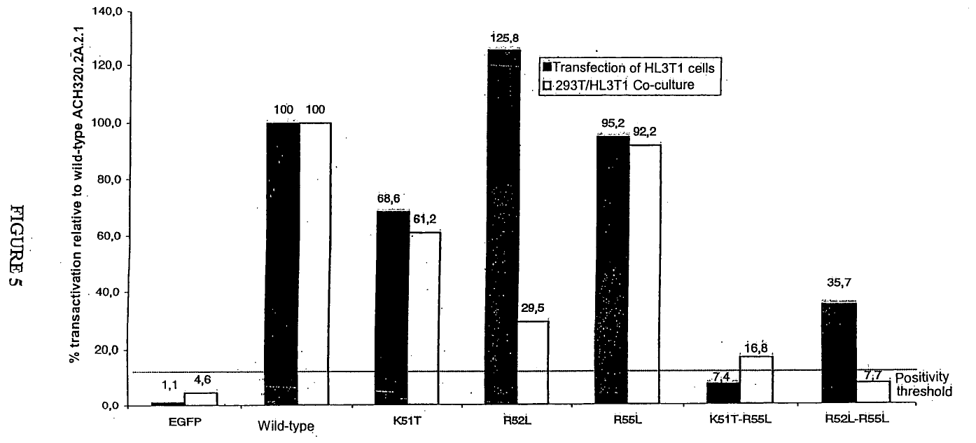

Different studies have shown that the total number of basic residues in the basic Tat domain plays a role in the capacity of the Tat protein, secreted by infected cells present in the extracellular medium, to be internalized by non-infected cells, a phenomenon also called transduction. The inventors evaluated the transduction capacity of the constructions containing mutations in this basic domain. A co-culture test was carried out between producer cells and effector cells. The 293T cells were transfected with 3 μg of the pEGFP-TEX constructions using the calcium phosphate technique (23). 24 hours after transfection, the transfected 293T cells were trypsinized and co-cultivated with 2.5×10⁵ HL3T1 cells for another 48 hours in the presence of 100 μM chloroquinine. The cells were then harvested and lysed in TEN before evaluation of the CAT activity, as described previously. Because this system depends on both the effectiveness of the transduction and the transactivating activity, the inventors started from the premise that a drastic change in the transduction capacity would be reflected in a significant difference between the activity measured after direct transfection and the activity measured after co-culture, compared with a standardized positive control. This is the reason why all the data are represented as percentages of the transactivating activity of the wild-type protein of the isolate ACH320.2A.2.1 and why the data obtained from the transfection of the HL3T1 cells and the co-culture of the 293T/HL3T1 cells are compared for each construction. As shown in FIG. 5, the R55L mutation does not significantly alter the transduction capacity of the protein. The mutation R52L significantly reduces the transduction capacity of the protein (5-fold reduction in the CAT activity between the transfected HL3T1 cells with this construction and the HL3T1 cells co-cultivated with the 293T cells expressing pEGFP-TEX-R52L). The double mutant R52L-R55L shows a complete loss of its transduction capacity, as shown by the background noise of the CAT activity measured after co-culture. The results obtained both with the R52L and R52L-R55L mutants suggest that Tat's transduction capacity is correlated with the number of arginine residues in the basic domain (27). It would seem that residue R55 is less important for the Tat's transduction capacity than residue R52. Therefore, the localization of the arginine residues could also play a role in the complete transduction mechanism.

### EXAMPLE 5

### Cloning of Cell Lines Transfected with the Nucleotide Sequences of the Invention

Numerous functional tests on the regulation of cell genes by the Tat protein of HIV-1 involve the use of cell lines expressing this protein in constitutive manner. The Inventors therefore established cell lines expressing the different Tat-protein mutants. In order to do this, HeLa cells were seeded at 2.5×10⁵ cells per well of a 6-well plate then transfected the next day with 2 μg of DNA coding for the various Tat mutants using the reagent Exgen 500 (marketed by Euromedex). In order to carry out a biological cloning of these transfected lines, the cells were trypsinized and counted 3 days after transfection, then seeded at a concentration of 3 to 30 cells per well in flat-bottomed 96-well plates, at a rate of 3 to 5 96-well plates per transfection (for a total of 288 to 480 wells per transfection). The culture in 96-well plates was then carried out for 15 days in the presence of 500 μg/ml of geneticin (Geneticin Sulfate, Gibco-BRL). After 15 days, the wells in which the cells were still alive and had multiplied notably were considered as positive. In standard manner, a biological cloning was considered successful when each 96-well plate from the same transfection contained less than 10 positive wells per plate. From 3 to 15 positive wells per transfection were then amplified in the presence of geneticin for 6 passages in order to obtain a sufficient quantity of cells for freezing. On the sixth passage, the expression of the Tat protein was verified by immunotransfer (western blot) for each clone.

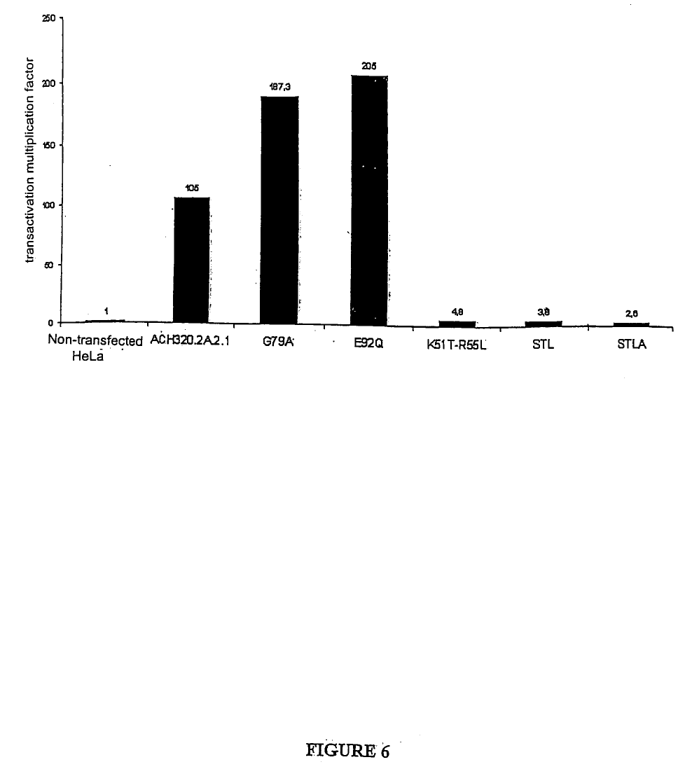

After verification of the expression of Tat by immunotransfer in the cell lines thus generated, the Inventors used a plasmid construction containing the CAT gene dependent on the viral HIV promoter (LTR-CAT construction) in order to transfect the different clones of cell lines thus obtained. It was thus possible to show that the stable expression of the Tat mutants in this cell line did not modify their transactivating activity (FIG. 6).

### EXAMPLE 6

### Mutant in Position 58

The single mutant S58A is obtained using as matrix the cDNA of the wild-type Tat gene of the strain ACH.320.2A.2.1 and as primer pair the pair S58A (M5′)/S58A (M3′). However, other HIV-1 strains exist which naturally carry an alanine in position 58 and which have all the properties of a functional Tat (nuclear localization, transactivation etc.) such as for example the strain HXB2. Such a mutation S58A on the strain ACH320.2A.2.1 does not modify the behaviour of the protein and does not make it possible to achieve the detoxification of Tat.

TABLE 1

Sequence

| | |
|---|---|
| End primers, 1st cycle | |
| E5′ | 5′- GAA TTC ATG GAG CCA GTA GAT C- 3′ |
| E3′ | 5′- AGA TCT CTA ATC GAC CGG ATC- 3′ |
| End primers, 2nd cycle | |
| EcoR I | 5′- AAA GAA TTC ATG GAG CCA GTA GAT CC- 3′ |
| E6854 | 5′- AAA GAT CTC TAA TCG ACC GGA TCT GTC TCT GTC TC- 3′ |
| Sal I | 5′- AAG TCG ACC TAA TCG ACC GGA TCT GTC TCT GTC TC- 3′ |
| Internal primers | |
| W11F (M5′) | 5′- CCA GTA GAT CCT AAA CTA GAG CCC TTC AAG CAT CCA G-3′ |
| C27S (MS′) | 5′- ACA ATT GCT ATT CGA AAA AGT G- 3′ |
| C27S (M3′) | 5′- CAC TTT TTC GAA TAG CAA TTG T- 3′ |
| K50R (M5′) | 5′- ATC TCA TAT GGC AGG CGG AAG -3′ |
| K50R (M3′) | 5′- CTT CCG CCT GCC ATA TGA GAT -3′ |
| K51T (M5′) | 5′- GGC AGG AAG ACC CGG AGA CAG C- 3′ |
| K51T (M3′) | 5′- GCT GTC TCC GGG TCT TCC TGC C- 3′ |
| R52L (M5′) | 5′- GGC AGG AAG AAG CTT AGA CAG CGA CGA AGA TC -3′ |
| R52L (M3′) | 5′- GAT CTT CGT CGC TGT CTA AGC TTC TTC CTG CC- 3′ |
| R55L (M5′) | 5′- GGC AGG AAG AAG CGG AGA CAG CTG CGA AGA TC- 3′ |
| R55L (M3′) | 5′- GAT CTT CGC AGC TGT CTC CGC TTC TTC CTG CC- 3′ |
| R57L (M5′) | 5′- GAC AGC GAC GAC TAT CTC CTC AAG AC -3′ |
| R57L (M3′) | 5′- GTC TTG AGG AGA TAG TCG TCG CTG TC- 3′ |
| G79A (MS′) | 5′- CAG CCC CGA GCG GAT CCG ACA GG- 3′ |
| G79A (M3′) | 5′- CCT GTC GGA TCC GCT CGG GGC TG- 3′ |
| K89L (M3′) | 5′- CTG TCT CTG TCT CTC TCT CCA CCT TAA GCT TCG ATT CC- 3′ |
| E92Q (M3′) | 5′- CTG TCT CTG TCT CTC TTT GCA CCT TCT TCT TCG AAT CC- 3′ |
| R52L-R55L (M5′) | 5′- GGC AGG AAG AAG CTT AGA CAG CTG CGA AGA TC - 3′ |
| R52L-R55L (M3′) | 5′- GAT CTT CGC AGC TGT CTA AGC TTC TTC CTG CC - 3′ |
| R55L-R57L (M5′) | 5′- GAA GCG GAG ACA GCT GCG ACT ATC TCC TCA AGA C -3′ |
| R55L-R57L (M3′) | 5′- GTC TTG AGG AGA TAG TCG CAG CTG TCT CCG CTT C -3′ |
| S58A (M5′) | 5′- GAC AGC GAC GAA GAG CAC CTC AAG ACA GT -3′ |
| S58A (M3′) | 5′- ACT GTC TTG AGG TGC TCT TCG TCG CTG TC -3′ |

### REFERENCES

1. Albini, A., G. Barillari, R. Benelli, R. C. Gallo, and B. Ensoli. 1995. Angiogenic properties of human immunodeficiency virus type 1 Tat protein. Proc Natl Acad Sci USA. 92:4838-4842.

2. Albini, A., R. Soldi, D. Giunciuglio, E. Giraudo, R. Benelli, L. Primo, D. Noonan, M. Salio, G. Camussi, W. Rockl, and F. Bussolino. 1996. The angiogenesis induced by HIV-1 tat protein is mediated by the Flk-1/KDR receptor on vascular endothelial cells. Nat Med. 2:1371-5.

3. Bartz, S. R., and M. Emerman. 1999. Human Immunodeficiency Virus type 1 Tat induces apoptosis and increases sensitivity to apoptotic signals by up-regulating FLICE/Caspase-8. J Virol. 73:1956-1963.

4. Cafaro, A., A. Caputo, C. Fracasso, M. T. Maggiorella, D. Goletti, S. Baroncelli, M. Pace, L. Sernicola, M. L. Koanga-Mogtomo, M. Beti, A. Borsetti, R. Belli, L. Akerblom, F. Corrias, S. Butto, J. Heeney, P. Verani, F. Titti, and B. Ensoli. 1999. Control of SHIV-89.6P-infection of cynomolgus monkeys by HIV-1 Tat protein vaccine. Nat Med. 5:643-650.

5. Cafaro, A., A. Caputo, M. T. Maggiorella, S. Baroncelli, C. Fracasso, M. Pace, A. Borsetti, L. Sernicola, D. R. Negri, P. Ten Haaft, M. Betti, Z. Michelini, I. Macchia, E. Fanales-Belasio, R. Belli, F. Corrias, S. Butto, P. Verani, F. Titti, and B. Ensoli. 2000. SHIV89.6P pathogenicity in cynomolgus monkeys and control of viral replication and disease onset by human immunodeficiency virus type 1 Tat vaccine. J Med Primatol. 29:193-208.

6. Cafaro, A., F. Titti, C. Fracasso, M. T. Maggiorella, S. Baroncelli, A. Caputo, D. Goletti, A. Borsetti, M. Pace, E. Fanales-Belasio, B. Ridolfi, D. R. Negri, L. Sernicola, R. Belli, F. Corrias, I. Macchia, P. Leone, Z. Michelini, P. ten Haaft, S. Butto, P. Verani, and B. Ensoli. 2001. Vaccination with DNA containing tat coding sequences and unmethylated CpG motifs protects cynomolgus monkeys upon infection with simian/human immunodeficiency virus (SHIV89.6P). Vaccine. 19:2862-77.

7. Cullen, B. R. 1992. Mechanism of action of regulatory proteins encoded by complex retroviruses. Microbiol Rev. 56:375-394.

8. Felber, B. K., and G. N. Pavlakis. 1988. A quantitative bioassay for HIV-1 based on transactivation. Science. 239:184-187.

9. Frankel, A. D., and C. O. Pabo. 1988. Cellular uptake of the tat protein from human immunodeficiency virus. Cell. 55:1189-1193.

10. Gringeri, A., E. Santagostino, M. Muca-Perja, H. The Buanec, B. Bizzini, A. Lachgar, J. F. Zagury, J. Rappaport, A. Burny, R. C. Gallo, and D. Zagury. 1999. Tat toxoid as a component of a preventive vaccine in seronegative subjects. J. Acquir. Immune Defic. Syndr. Hum Retrovirol. 20:371-375.

11. Groenink, M., A. C. Andeweg, R. A. Fouchier, S. Broersen, R. C. van der Jagt, H. Schuitemaker, R. E. of Goede, M. L. Bosch, H. G. Huisman, and M. Tersmette. 1992. Phenotype-associated env gene variation among eight related human immunodeficiency virus type 1 clones: evidence for in vivo recombination and determinants of cytotropism outside the V3 domain. J Virol. 66:6175-6180.

12. Guillon, C., F. Bedin, R. A. M. Fouchier, H. Schuitemaker, and R. A. Gruters. 1995. Completion of nucleotide sequences of non-syncytium-inducing and syncytium-inducing HIV type 1 variants isolated from the same patient. AIDS Res Hum Retroviruses. 11:1537-1538.

13. Hauber, J., M. H. Malim, and B. R. Cullen. 1989. Mutational analysis of the conserved basic domain of human immunodeficiency virus Tat protein. J Virol. 63:1181-1187.

14. Helland, D. E., J. L. Welles, A. Caputo, and W. A. Haseltine. 1991. Transcellular transactivation by the human immunodeficiency virus type 1 Tat protein. J Virol. 65:4547-4549.

15. Higuchi, R. 1990. Recombinant PCR, p. 177-183. In M. A. Innis, Gelfand, D. H., Sninsky, J. J. and White, T. J. (ed.), PCR protocols: a guide to methods and applications. Academic Press, Inc., San Diego, Calif.

16. Howeroft, T. K., K. Strebel, M. A. Martin, and D. S. Sinder. 1993. Repression of MHC class I gene promoter activity by two-exon Tat of HIV. Science. 260:1320-1322.

17. Jeang, K. -T. 1996. HIV-1 Tat: structure and function, p. 3-18. In G. Myers and B. Foley and J. W. Mellors and B. Korber and K. T. Jeang and S. Wain-Hobson (ed.), Human retroviruses and AIDS 1996: a compilation and analysis of nucleic acid and amino acid sequences. Los Alamos National Laboratory, Los Alamos.

18. Jeang, K. T., H. Xiao, and E. A. Rich. 1999. Multifaceted activities of HIV-1 transactivator of transcription, Tat. J Biol Chem. 274:28837-28840.

19. Meyaard, L., S. A. Otto, H. Schuitemaker, and F. Miedema. 1992. Effects of HIV-1 Tat protein on human T-cell proliferation. Eur J Immunol. 22:2729-2732.

20. Mitola, S., R. Soldi, I. Zanon, L. Barra, M. I. Gutierrez, B. Berkhout, M. Giacca, and F. Bussolino. 2000. Identification of specific molecular structures of human immunodeficiency virus type 1 Tat relevant for its biological effects on vascular endothelial cells. J Virol. 74:344-53.

21. Osterhaus, A. D. M. E., C. A. van Baalen, R. A. Gruters, M. Schutten, C. H. Siebelink, E. G. Hulskotte, E. J. Tijhaar, R. E. Randall, G. van Amerongen, A. Fleuchaus, V. Erfle, and G. Sutter. 1999. Vaccination with Rev and Tat against AIDS. Vaccine. 17:2713-2714.

22. Ott, M., S. Emiliani, C. Van Lint, G. Herbein, J. Lovett, N. Chirmule, T. McCloskey, S. Pahwa, and E. Verdin. 1997. Immune hyperactivation of HIV-1-infected T cells mediated by Tat and the CD28 pathway. Science. 275:1481-1485.

23. Sambrook, J., E. F. Fritsch, and T. Maniatis. 1989. Molecular cloning. A laboratory manual. Cold Spring Harbor Laboratory Press, Cold Spring Harbor, N.Y.

24. Seed, B., and J. Sheen. 1988. A simple phase extraction assay for chloramphenicol acetyltransferase activity. Gene. 67:271-277.

25. Stauber, R. H., and G. N. Pavlakis. 1998. Intracellular trafficking and interactions of HIV-1 Tat protein. Virology. 252:126-136.

26. van Baalen, C. A., O. Pontesilli, R. C. Huisman, A. M. Geretti, M. R. Klein, F. of Wolf, F. Miedema, R. A. Gruters, and A. D. M. E. Osterhaus. 1997. Human immunodeficiency virus type 1 Rev- and Tat-specific cytotoxic T lymphocyte frequencies inversely correlate with rapid progression to AIDS. J Gen Virol. 78:1913-1918.

27. Wender, P. A., D. J. Mitchell, K. Pattabiraman, E. T. Pelkey, L. Steinman, and J. B. Rothbard. 2000. The design, synthesis, and evaluation of molecules that enable or enhance cellular uptake: Peptoid molecular transporters. Proc Natl Acad Sci USA. 97:13003-13008.

28. Westendorp, M. O., R. Frank, C. Oschenbauer, K. Stricker, J. Dhein, H. Walczak, K. M. Debatin, and P. H. Krammer. 1995. Sensitization of T cells to CD95-mediated apoptosis by HIV-1 Tat and gp120. Nature. 375:497-500.

29. Westendorp, M. O., M. Li-Weber, R. W. Frank, and P. H. Krammer. 1994. Human immunodeficiency virus type 1 Tat upregulates interleukin-2 secretion in activated T cells. J. Virol. 68:4177-4185.

30. Zauli, G., D. Gibellini, D. Milani, M. Mazzoni, P. Borgatti, M. La Placa, and S. Capitani. 1993. Human immunodeficiency virus type 1 Tat protein protects lymphoid, epithelial, and neuronal cell lines from death by apoptosis. Cancer Res. 53:4481-4485.

31. Mayhood T, Kaushik N, Pandey P K, Kashanchi F, Deng L, Pandey V N. 2000. Inhibition of Tat-mediated transactivation of HIV-1 LTR transcription by polyamide nucleic acid targeted to TAR hairpin element. Biochemistry, 39, 11532-11539.

32. Tyagi M., Rusnati M., Presta M., Giacca M. 2001. Internalization of HIV-1 tat requires cell surface heparan sulfate proteoglycans. J. Biol. Chem., 276, 3254-3261.

33. Stauber R H, Pavlakis G N. 1998. Intracellular trafficking and interactions of HIV-1 Tat protein. Virology, 252, 126-136.

34. Endo S., Kubota S., Siomi H., Adachi A., Oroszlan S., Maki M., Hatanaka M. 1989. A region of basic amino-acid cluster in HIV-1 Tat protein is essential for trans-acting activity and nucleolar localization. Virus Genes, 3, 99-110.

1. Detoxified and immunogenic mutant of the Tat protein of the HIV-1 virus, characterized in that it comprises at least two mutations in regions 4 and/or 5 of the wild-type Tat protein, and in that, when the mutation is in the region of domain 4, it is in the part delimited by the amino acid in position 49 to the amino acid in position 57, and in that, when the mutation is in domain 5, it is either in the RGD motif, or in the region 88-92, preferably in positions 89 and/or 92, the mutations being mutations by substitution of one amino acid by another.

2. Mutant according to claim 1, characterized in that it comprises at least one mutation in region 4.

3. Mutant according to claim 1, characterized in that the mutations in domains 4 and/or 5 are capable of conferring at least one of the following properties:
the cancellation of the transcellular effect of the wild-type Tat protein,
the alteration of the nuclear localization of the wild-type Tat protein.

4. Mutant according to claim 1, characterized in that it comprises an additional mutation capable of conferring a loss of the transactivating activity of the wild-type Tat protein.

5. Mutant according to claim 1, characterized in that it comprises a mutation in the N-terminal region of domain 4 of the wild-type Tat protein in the part delimited by the amino acid in position 49 to the amino acid in position 55.

6. Mutant according to claim 1, characterized in that it comprises a mutation in domain 2 of the wild-type Tat protein, in particular the replacement of any one of the cysteines, advantageously by a serine.

7. Mutant according to claim 1, characterized in that it comprises at least one of the following mutations:
replacement in position 27 of a cysteine by a serine,
replacement in position 51 of a lysine by a threonine,
replacement in position 52 of an arginine by a leucine,
replacement in position 55 of an arginine by a leucine,
replacement in position 57 of an arginine by a leucine,
replacement in position 79 of a glycine by an alanine,
replacement in position 89 of a lysine by a leucine,
replacement in position 92 of a glutamic acid by a glutamine.

8. Mutant according to claim 1, characterized in that it is chosen from the mutants having two mutations as indicated hereafter, each of the mutations being represented by a triplet: letter-figure-letter, the figure of which indicates the position of the mutated amino acid, the letter preceding the figure corresponds to the amino acid to which the mutation relates and the letter following the figure corresponds to the amino acid replacing the amino acid preceding the figure:

| | |
|---|---|
| K51T-R52L | (SEQ ID NO: 2) |
| K51T-R55L | (SEQ ID NO: 3) |
| K51T-R57L | (SEQ ID NO: 4) |
| K51T-G79A | (SEQ ID NO: 5) |
| K51T-K89L | (SEQ ID NO: 6) |
| K51T-E92Q | (SEQ ID NO: 7) |
| R52L-R55L | (SEQ ID NO: 8) |
| R52L-R57L | (SEQ ID NO: 9) |
| R52L-G79A | (SEQ ID NO: 10) |
| R52L-K89L | (SEQ ID NO: 11) |
| R52L-E92Q | (SEQ ID NO: 12) |
| R55L-R57L | (SEQ ID NO: 13) |
| R55L-G79A | (SEQ ID NO: 14) |
| R55L-K89L | (SEQ ID NO: 15) |
| R55L-E92Q | (SEQ ID NO: 16) |
| R57L-G79A | (SEQ ID NO: 17) |
| R57L-K89L | (SEQ ID NO: 18) |
| R57L-E92Q | (SEQ ID NO: 19) |
| G79A-K89L | (SEQ ID NO: 20) |
| G79A-E92Q | (SEQ ID NO: 21) |
| K89L-E92Q | (SEQ ID NO: 22). |

9. Mutant according to claim 8, characterized in that it is chosen from the following mutants:

| | |
|---|---|
| K51T-R55L | (SEQ ID NO: 3) |
| R52L-R55L | (SEQ ID NO: 8) |
| R52L-G79A | (SEQ ID NO: 10) |
| R55L-R57L | (SEQ ID NO: 13) |
| G79A-K89L | (SEQ ID NO: 20). |

10. Mutant according to claim 1, characterized in that it is chosen from the mutants having three mutations as indicated hereafter, each of the mutations being represented by a triplet: letter-figure-letter, the figure of which indicates the position of the mutated amino acid, the letter preceding the figure corresponds to the amino acid to which the mutation relates and the letter following the figure corresponds to the amino acid replacing the amino acid preceding the figure:

| | |
|---|---|
| C27S-K51T-R52L | (SEQ ID NO: 23) |
| C27S-K51T-R55L | (SEQ ID NO: 24) |
| C27S-K51T-R57L | (SEQ ID NO: 25) |
| C27S-K51T-G79A | (SEQ ID NO: 26) |
| C27S-K51T-K89L | (SEQ ID NO: 27) |
| C27S-K51T-E92Q | (SEQ ID NO: 28) |
| C27S-R52L-R55L | (SEQ ID NO: 29) |
| C27S-R52L-R57L | (SEQ ID NO: 30) |
| C27S-R52L-G79A | (SEQ ID NO: 31) |
| C27S-R52L-K89L | (SEQ ID NO: 32) |
| C27S-R52L-E92Q | (SEQ ID NO: 33) |
| C27S-R55L-R57L | (SEQ ID NO: 34) |
| C27S-R55L-G79A | (SEQ ID NO: 35) |
| C27S-R55L-K89L | (SEQ ID NO: 36) |
| C27S-R55L-E92Q | (SEQ ID NO: 37) |
| C27S-R57L-G79A | (SEQ ID NO: 38) |
| C27S-R57L-K89L | (SEQ ID NO: 39) |
| C27S-R57L-E92Q | (SEQ ID NO: 40) |
| C27S-G79A-K89L | (SEQ ID NO: 41) |
| C27S-G79A-E92Q | (SEQ ID NO: 42) |
| C27S-K89L-E92Q | (SEQ ID NO: 43). |

11. Mutant according to claim 10, characterized in that it is chosen from the following mutants:

| | |
|---|---|
| C27S-K51T-R55L | (SEQ ID NO: 24) |
| C27S-R52L-R55L | (SEQ ID NO: 29) |
| C27S-R52L-G79A | (SEQ ID NO: 31). |

12. Mutant according to claim 1, characterized in that it is chosen from the mutants having four mutations as indicated hereafter, each of the mutations being represented by a triplet: letter-figure-letter, the figure of which indicates the position of the mutated amino acid, the letter preceding the figure corresponds to the amino acid to which the mutation relates and the letter following the figure corresponds to the amino acid replacing the amino acid preceding the figure:

| | |
|---|---|
| C27S-K51T-R52L-G79A | (SEQ ID NO: 44) |
| C27S-K51T-R52L-K89L | (SEQ ID NO: 45) |
| C27S-K51T-R52L-E92Q | (SEQ ID NO: 46) |
| C27S-K51T-R55L-G79A | (SEQ ID NO: 47) |
| C27S-K51T-R55L-K89L | (SEQ ID NO: 48) |
| C27S-K51T-R55L-E92Q | (SEQ ID NO: 49) |
| C27S-K51T-R57L-G79A | (SEQ ID NO: 50) |
| C27S-K51T-R57L-K89L | (SEQ ID NO: 51) |
| C27S-K51T-R57L-E92Q | (SEQ ID NO: 52) |
| C27S-K51T-G79A-K89L | (SEQ ID NO: 53) |
| C27S-K51T-G79A-E92Q | (SEQ ID NO: 54) |
| C27S-K51T-K89L-E92Q | (SEQ ID NO: 55) |
| C27S-R52L-G79A-K89L | (SEQ ID NO: 56) |
| C27S-R52L-G79A-E92Q | (SEQ ID NO: 57) |
| C27S-R52L-K89L-E92Q | (SEQ ID NO: 58) |
| C27S-R52L-R55L-G79A | (SEQ ID NO: 59) |
| C27S-R52L-R55L-K89L | (SEQ ID NO: 60) |
| C27S-R52L-R55L-E92Q | (SEQ ID NO: 61) |
| C27S-R52L-R57L-G79A | (SEQ ID NO: 62) |
| C27S-R52L-R57L-K89L | (SEQ ID NO: 63) |
| C27S-R52L-R57L-E92Q | (SEQ ID NO: 64) |
| C27S-R55L-G79A-K89L | (SEQ ID NO: 65) |
| C27S-R55L-G79A-E92Q | (SEQ ID NO: 66) |
| C27S-R55L-K89L-E92Q | (SEQ ID NO: 67) |
| C27S-R55L-R57L-G79A | (SEQ ID NO: 68) |
| C27S-R55L-R57L-K89L | (SEQ ID NO: 69) |
| C27S-R55L-R57L-E92Q | (SEQ ID NO: 70) |
| C27S-R57L-G79A-K89L | (SEQ ID NO: 71) |
| C27S-R57L-G79A-E92Q | (SEQ ID NO: 72) |
| C27S-R57L-K89L-E92Q | (SEQ ID NO: 73) |
| C27S-G79A-K89L-E92Q | (SEQ ID NO: 74). |

13. Mutant according to claim 12, characterized in that it is chosen from the following mutants:

| | |
|---|---|
| C27S-K51T-R55L-G79A | (SEQ ID NO: 47) |
| C27S-K51T-R55L-K89L | (SEQ ID NO: 48) |
| C27S-K51T-R55L-E92Q | (SEQ ID NO: 49) |
| C27S-R52L-R55L-G79A | (SEQ ID NO: 59). |

14. Mutant according to claim 1, characterized in that it is chosen from the mutants having five mutations as indicated hereafter, each of the mutations being represented by a triplet: letter-figure-letter, the figure of which indicates the position of the mutated amino acid, the letter preceding the figure corresponds to the amino acid to which the mutation relates and the letter following the figure corresponds to the amino acid replacing the amino acid preceding the figure:

| | |
|---|---|
| C27S-K51T-G79A-K89L-E92Q | (SEQ ID NO: 75) |
| C27S-K51T-R52L-R55L-G79A | (SEQ ID NO: 76) |
| C27S-K51T-R52L-R55L-K89L | (SEQ ID NO: 77) |
| C27S-K51T-R52L-R55L-E92Q | (SEQ ID NO: 78) |
| C27S-K51T-R52L-R57L-G79A | (SEQ ID NO: 79) |
| C27S-K51T-R52L-R57L-K89L | (SEQ ID NO: 80) |
| C27S-K51T-R52L-R57L-E92Q | (SEQ ID NO: 81) |
| C27S-K51T-R52L-G79A-K89L | (SEQ ID NO: 82) |
| C27S-K51T-R52L-G79A-E92Q | (SEQ ID NO: 83) |
| C27S-K51T-R52L-K89L-E92Q | (SEQ ID NO: 84) |
| C27S-K51T-R55L-R57L-G79A | (SEQ ID NO: 85) |
| C27S-K51T-R55L-R57L-K89L | (SEQ ID NO: 86) |
| C27S-K51T-R55L-R57L-E92Q | (SEQ ID NO: 87) |
| C27S-K51T-R55L-G79A-K89L | (SEQ ID NO: 88) |
| C27S-K51T-R55L-G79A-E92Q | (SEQ ID NO: 89) |
| C27S-K51T-R55L-K89L-E92Q | (SEQ ID NO: 90) |
| C27S-K51T-R57L-G79A-K89L | (SEQ ID NO: 91) |
| C27S-K51T-R57L-G79A-E92Q | (SEQ ID NO: 92) |
| C27S-K51T-R57L-K89L-E92Q | (SEQ ID NO: 93) |
| C27S-R52L-R55L-R57L-G79A | (SEQ ID NO: 94) |
| C27S-R52L-R55L-R57L-K89L | (SEQ ID NO: 95) |
| C27S-R52L-R55L-R57L-E92Q | (SEQ ID NO: 96) |
| C27S-R52L-R55L-G79A-K89L | (SEQ ID NO: 97) |
| C27S-R52L-R55L-G79A-E92Q | (SEQ ID NO: 98) |
| C27S-R52L-R55L-K89L-E92Q | (SEQ ID NO: 99) |
| C27S-R52L-R57L-G79A-K89L | (SEQ ID NO: 100) |
| C27S-R52L-R57L-G79A-E92Q | (SEQ ID NO: 101) |
| C27S-R52L-R57L-K89L-E92Q | (SEQ ID NO: 102) |
| C27S-R52L-G79A-K89L-E92Q | (SEQ ID NO: 103) |
| C27S-R55L-R57L-G79A-K89L | (SEQ ID NO: 104) |
| C27S-R55L-R57L-G79A-E92Q | (SEQ ID NO: 105) |
| C27S-R55L-R57L-K89L-E92Q | (SEQ ID NO: 106) |
| C27S-R55L-G79A-K89L-E92Q | (SEQ ID NO: 107) |
| C27S-R57L-G79A-K89L-E92Q | (SEQ ID NO: 108). |

15. Mutant according to claim 14, characterized in that it is chosen from the following mutants:

| | |
|---|---|
| C27S-K51T-R55L-G79A-K89L | (SEQ ID NO: 88) |
| C27S-K51T-R55L-G79A-E92Q | (SEQ ID NO: 89). |

16. Nucleotide sequences coding for one of the mutants according to claim 1.

17. Cell line transfected with a nucleotide sequence according to claim 16.

18. Antibodies directed against one of the mutants according to claim 1, not recognizing domain D1 of the wild-type protein.

19. Antibodies according to claim 18, recognizing the wild-type protein.

20. Antibodies according to claim 18, not recognizing the wild-type protein.

21. Pharmaceutical composition, in particular a vaccine, containing as active ingredient at least one of the mutants according to claim 1, placed under the control of elements necessary to a constitutive expression of one of the mutants, in combination with a pharmaceutically suitable vehicle.

22. Diagnostic composition for the detection and/or quantification of HIV-1 virus comprising at least one mutant as defined in claim 1.

23. Process for the detection and/or quantification of the HIV-1 virus in a biological sample taken from an individual capable of being infected with HIV-1, such as plasma, serum or tissue, characterized in that it comprises stages consisting of:
bringing said biological sample into contact with a diagnostic composition comprising a mutant as defined in claim 1, under predetermined conditions which allow, if necessary, the formation of antibody/antigen complexes between the mutant defined above and antibodies directed against the wild-type Tat protein or between the antibodies defined above and the wild-type Tat protein, and
detecting and/or quantifying the formation of said complexes by any appropriate means.

24. Method for the in vitro diagnosis of the HIV-1 virus in a biological specimen or sample, which comprises using at least one mutant as defined in claim 1.

25. Method for the preparation of a vaccine composition, which comprises using at least one mutant as defined in claim 1.
